(19) United States
(12) Patent Application Publication
(10) Pub. No.: US 2019/0336583 A1
(43) Pub. Date: Nov. 7, 2019

(54) COMPOSITIONS AND METHODS FOR TREATING IRON OVERLOAD

(71) Applicant: La Jolla Pharmaceutical Company, San Diego, CA (US)

(72) Inventors: George Tidmarsh, Portola Valley, CA (US); Lakhmir Chawla, San Diego, CA (US)

(21) Appl. No.: 16/478,998

(22) PCT Filed: Jan. 18, 2018
(86) PCT No.: PCT/US2018/014241

§ 371 Date: Jul. 18, 2019

Related U.S. Application Data
(60) Provisional application No. 62/554,115, filed on Sep. 5, 2017.
(60) Provisional application No. 62/454,322, filed on Feb. 3, 2017.
(60) Provisional application No. 62/447,710, filed on Jan. 18, 2017.

(51) Int. Cl. A61K 38/22; A61K 9/00; A61P 7/06

(52) U.S. Cl. CPC A61K 38/22; A61P 7/06; A61K 9/0019

(57) ABSTRACT

The present disclosure relates to the use of hepcidin, mini-hepcidin, or a hepcidin analogue in therapeutic methods for the treatment and/or prevention of acquired iron overload or other conditions for which iron redistribution or sequestration is helpful.

### RELATED APPLICATIONS

This application claims the benefit of priority to U.S. Provisional Patent Application Ser. No. 62/447710, filed on Jan. 18, 2017, U.S. Provisional Patent Application Ser. No. 62/454322, filed on Feb. 3, 2017, and U.S. Provisional Patent Application Ser. No. 62/554115, filed on Sep. 5, 2017, each of which are herein incorporated by reference in their entireties.

### BACKGROUND

Iron is an essential element required for growth and survival of almost every organism. In mammals, the iron balance is primarily regulated at the level of duodenal absorption of dietary iron. Following absorption, ferric iron is loaded into apo-transferrin in the circulation and transported to the tissues, including erythroid precursors, where it is taken up by transferrin receptor-mediated endocytosis. Reticuloendothelial macrophages play a major role in the recycling of iron from the degradation of hemoglobin of senescent erythrocytes, while hepatocytes contain most of the iron stores of the organism in ferritin polymers.

Patients who require frequent blood transfusions, such as those with severe anemia or thalassemia, are at risk of developing iron overload (referred to in such cases as “acquired iron overload”). Specifically, a single unit of blood contains 250 times more iron than the body's daily metabolic requirement. Since the body is unable to effectively secrete iron through the urine, transfusion patients accumulate a large excess of iron that cannot be stored in the liver. After as few as ten blood transfusions, the signs and symptoms of iron overload can emerge, including joint pain, fatigue, general weakness, unexplained weight loss, and stomach pain. Later signs of iron overload can include arthritis, liver disease, diabetes, heart abnormalities, and skin discoloration.

Phlebotomy and iron chelators are commonly used to treat iron overload. However, patients with iron overload due to transfusion-dependent conditions may not tolerate phlebotomy. For these patients, iron chelation is the recommended course of action. Iron chelators are designed to specifically bind and remove iron from the blood. There are a number of these drugs, but in the US, there are just two approved for use in patients receiving frequent blood transfusions. Deferoxamine (DFO) has been in widespread clinical use since the late 1970s and has provided evidence that chelation is an effective therapy. DFO is a hexadentate chelator with a high and selective affinity for iron. The drug is administered as long infusions because the plasma half-life is short and it is not orally bioavailable. The second approved drug for iron overload is deferasirox. The drug is an oral iron chelator for the treatment of transfusion-dependent iron overload and non-transfusion-dependent thalassemia. Although they can be effective at managing iron overload, the above chelators are associated with serious liver and kidney toxicity. Additionally, chelator therapies are directed to reducing circulating free iron. But free iron is a small component of total iron, as most somatic iron is reversibly bound by transferrin or contained in the red blood cell mass and organs/tissues. In individuals with normal iron homeostasis, transferrin binds free iron with high avidity between about 25-45%. When transferrin saturation drops below 20-25%, iron is restricted for physiological use. Above 50-70%, the transferrin cannot retain all the iron in a bound state and some is released as free iron. Chelators are therefore limited in their rate of iron clearance. Thus, there is a clear need for safer and/or additional alternatives for managing acquired iron overload and for reducing circulating or total body iron. The current invention provides a way to safely sequester and/or redistribute iron in the body to reduce free iron and iron overload in the tissues and organs.

### SUMMARY

While iron is critical for many physiological functions, iron can lead to oxidative damage of tissues, increased risk of infection, and iron overload in organs and tissues. It has been discovered that even in conditions where iron is not a causative agent of a disorder, it may be a mediator of ill effects; and managing or selectively reducing transferrin saturation and free iron stores by administration of hepcidin can treat, prevent, or ameliorate such conditions. Thus, the instant invention allows titratable management of free and transferrin-bound iron that cannot be done with current therapies for a variety of conditions where iron depletion or withholding may be useful, such as in organ/tissue reperfusion, acute kidney injury or vascular disorders, in endothelial or epithelial cells where iron mediates many physiological functions, disorders affecting bone marrow function that impact iron stores, etc.

The present disclosure relates to the use of hepcidin or mini-hepcidin in therapeutic methods for the treatment of acquired iron overload, such as the iron overload that is the product of blood transfusions (e.g., in patients who have anemia (such as aplastic anemia, hemolytic anemia, or sideroblastic anemia), thalassemia (e.g., hemoglobin E-beta thalassaemia (Hb E/β-thalassaemia) or hemoglobin E thalassemia), sickle cell disease, myelodysplastic syndrome, or who have undergone physical trauma). In some aspects, provided herein are methods for treating acquired iron overload in a subject by administering a composition comprising hepcidin or mini-hepcidin to the subject. In some embodiments, provided herein is a method for preventing iron overload in a subject who is undergoing a blood transfusion (e.g., a subject who has anemia (such as aplastic anemia, hemolytic anemia or sideroblastic anemia), thalassemia, sickle cell disease, myelodysplastic syndrome, or who has undergone physical trauma), comprising administering a composition comprising hepcidin or mini-hepcidin to the subject (e.g., before, during, or after the blood transfusion). In some aspects, provided herein are methods for treating and/or preventing a condition (e.g., iron overload resulting from a cardiovascular surgery, cardiopulmonary bypass, acute coronary syndrome, or sepsis) in a subject by administering a composition comprising hepcidin or mini-hepcidin to the subject according to any of the methods discussed herein. In some embodiments, the subject is undergoing cardiovascular surgery such as a cardiopulmonary bypass. In some embodiments, the subject has previously undergone cardiovascular surgery such as a cardiopulmonary bypass.

In further aspects, provided herein are methods of treating and/or preventing a condition, for example, insulin resistance, insulin insufficiency (diabetes), carotid artery lesion, chronic kidney disease, acute kidney injury, proteinuria, anti-glomerular basement membrane (anti-GMB) glomerulonephritis, minimal change disease (nephrotic syndrome), membrane nephropathy, autoimmune glomerulonephritis (e.g., immune complex induced glomerulonephritis), or conditions where the bone marrow is compromised (e.g., conditions in which compromised bone marrow can lead to acute increase in serum iron because the bone marrow is absorbing less iron), by administering a composition comprising hepcidin or mini-hepcidin to a subject. In certain such embodiments, the condition is caused or exacerbated by acquired iron overload in the subject.

In even further aspects, provided herein are methods of reducing total body iron in a subject having acquired iron overload by administering hepcidin or mini-hepcidin. In some such embodiments, provided herein are methods of reducing total body iron in a subject having acquired iron overload resulting from a blood transfusion (e.g., a subject who has anemia (such as aplastic anemia, hemolytic anemia or sideroblastic anemia), thalassemia, sickle cell disease, myelodysplastic syndrome, or who has undergone physical trauma), by administering a composition comprising hepcidin or mini-hepcidin to the subject (e.g., before, during, or after the blood transfusion).

In some aspects, provided herein are methods for reducing total body iron in a subject having acquired iron overload (e.g., iron overload resulting from a cardiovascular surgery, cardiopulmonary bypass, acute coronary syndrome, or sepsis) in a subject by administering a composition comprising hepcidin or mini-hepcidin to the subject according to any of the methods discussed herein. In some such embodiments, the subject is undergoing cardiovascular surgery such as a cardiopulmonary bypass. In other such embodiments, the subject has previously undergone cardiovascular surgery such as a cardiopulmonary bypass. In yet other such embodiments, the subject has a condition, for example, insulin resistance and insufficiency (diabetes), carotid artery lesion, chronic kidney disease, acute kidney injury, proteinuria, anti-glomerular basement membrane (anti-GMB) glomerulonephritis, minimal change disease (nephrotic syndrome), membrane nephropathy, or autoimmune glomerulonephritis (e.g., immune complex induced glomerulonephritis).

In some embodiments, an individual has total body iron within normal physiological ranges (e.g., the subject has transient iron overload or no iron overload). In other embodiments, an individual has a level of total body iron above normal physiological ranges. For example, in some embodiments, the subject has a total body iron content of about 40 to about 50 mg/kg prior to administering the composition. In other embodiments, the subject has iron overload (e.g., acquired iron overload). For example, the subject may have a total body iron content greater than about 50 mg/kg prior to administering the composition, such as greater than about 55 mg/kg, greater than about 60 mg/kg, greater than about 65 mg/kg, or greater than about 70 mg/kg.

### DETAILED DESCRIPTION

In some aspects, provided herein are methods for treating acquired iron overload in a subject by administering a composition comprising hepcidin or mini-hepcidin to the subject. In some aspects, provided herein are methods for reducing the serum iron concentration in a subject with acquired iron overload by administering a composition comprising hepcidin or mini-hepcidin to the subject. In some aspects, provided herein are methods for preventing iron overload in a subject who is undergoing a blood transfusion comprising administering a composition comprising hepcidin or mini-hepcidin to the subject (e.g., before, during or after the blood transfusion). Administering hepcidin or mini-hepcidin may comprise subcutaneous administration, such as subcutaneous injection. Alternatively, administering hepcidin or mini-hepcidin may comprise intravenous administration. The subject may have anemia (such as aplastic anemia, hemolytic anemia or sideroblastic anemia), thalassemia (e.g., hemoglobin E-beta thalassemia (Hb E/β-thalassemia) or hemoglobin E thalassemia), sickle cell disease, or myelodysplastic syndrome. In other embodiments, the subject may be experiencing or about to experience physical trauma (e.g., physical trauma (including surgical intervention) resulting in blood loss or need for or administration of a blood transfusion). The subject may have a tissue injury (e.g., a crush injury or a burn injury). Treatment of such patients with hepcidin or a mini-hepcidin can protect such subjects from iron-induced injury resulting from the injury or transfusion. In certain such embodiments, the subject may have acute kidney injury. In some aspects, provided herein are methods for treating and/or preventing a condition (e.g., iron overload resulting from cardiovascular surgery such as a cardiopulmonary bypass, acute coronary syndrome, or sepsis) in a subject by administering a composition comprising hepcidin or mini-hepcidin to the subject according to any of the methods discussed herein.

An aspect of the invention provides methods of treating and/or preventing insulin resistance, artery lesions, or kidney malfunctions, such as chronic kidney disease (CKD) or acute kidney injury (AKI). Accordingly, certain embodiments of the invention provide methods for treating and/or preventing a condition by administering a composition comprising hepcidin or mini-hepcidin to a subject. In some embodiments, the condition is, for example, insulin resistance and insufficiency (diabetes), carotid artery lesion, chronic kidney disease, acute kidney injury, proteinuria, anti-glomerular basement membrane (anti-GMB) glomerulonephritis, minimal change disease (nephrotic syndrome), membrane nephropathy, or autoimmune glomerulonephritis (e.g., immune complex induced glomerulonephritis). In certain such embodiments, the condition is caused by an iron overload in the subject.

Iron chelation therapy or iron-deficient diet ameliorates proteinuria and improves renal structure and function in animal models of anti-GMB glomerulonephritis, puromycin-induced MCD, membranous nephropathy, and immune complex induced glomerulonephritis. Accordingly, in some embodiments, the invention provides methods of treating and/or preventing a condition, for example, insulin resistance and insufficiency (diabetes), carotid artery lesion, chronic kidney disease, acute kidney injury, proteinuria, anti-glomerular basement membrane (anti-GMB) glomerulonephritis, minimal change disease (nephrotic syndrome), membrane nephropathy, or autoimmune glomerulonephritis (e.g., immune complex induced glomerulonephritis) by administering a composition comprising hepcidin or mini-hepcidin to a subject conjointly with an iron chelation therapy and/or an iron-deficient diet. In certain such embodiments, the condition is caused by an iron overload in the subject.

Iron chelation therapy is used to remove excess iron from a subject and reverse iron accumulation related problems. Iron chelation therapy comprises administering agents that capture non-transferrin-bound iron and labile plasma iron to reduce iron overload and prevent adverse consequences of iron overload. Iron chelation therapy involves sequestration of iron from the blood using a chelator, thereby reducing the total blood iron; however, merely sequestering iron from the blood may not always reduce the total body iron in a subject. Several iron chelation therapies are known in the art, some of which are summarized by Poggiali et al. (2012), An Update on Iron Chelation Therapy, Blood Transfusion; 10(4):411-422. The Poggiali et al. reference is herein incorporated by reference in its entirety, particularly, Table 1. Certain such iron chelation therapies include, Deferoxamine, Deferiprone, Deferasirox, α-ketohydroxypyridine analogue of Deferiprone, Deferitrin, 1-allyl-2-methyl-3-hydroxypyrid-4-one (LINAII), and deferitazole.

Additional iron chelating agents are described in United States Patent Application Publication No. 20120189551, which is incorporated by reference herein in its entirety. Particularly, such iron chelating agents include hydroxamic acids and derivatives thereof, N-hydroxyureas, 2-benzyl-1-naphthol, catechols, hydroxylamines, carnosol trolox C, catechol, naphthol, sulfasalazine, zyleuton, 5-hydroxyanthranilic acid and 4-(omega-arylalkyl)phenylalkanoic acids), imidazole-containing compounds (e.g., ketoconazole and itraconazole), phenothiazines, and benzopyran derivatives.

Administering any of the iron chelation therapies known in the art conjointly with administering a composition comprising hepcidin or mini-hepcidin is envisioned.

The term “total body iron” represents the total amount of iron present in a subject's body. A healthy human male has about 50 mg of iron per kg of body weight and a healthy human female has about 40 mg of iron per kg of body weight. A person skilled in the art can determine a healthy level of total body iron.

The term “total blood iron” represents the amount of iron present in a subject's blood. A healthy human male has about 60 to 170 μg of iron dL of serum and a healthy human female has about 30 to 126 μg of iron per dL of serum. A person skilled in the art can determine healthy levels of total blood iron in a subject.

Reducing total blood iron in a subject suffering from iron overload may address some of the adverse effects of iron overload; however, if the subject's total body iron is not reduced, certain adverse effects of iron overload may persist. Therefore, therapies that remove iron from a subject, for example, via urinary or fecal excretion, and thus reduce total body iron are provided.

Accordingly, provided herein are methods of reducing total body iron in a subject by administering hepcidin or mini-hepcidin to the subject, such as a subject having acquired iron overload. The acquired iron overload may result from a blood transfusion (e.g., the subject may have anemia (such as aplastic anemia, hemolytic anemia or sideroblastic anemia), thalassemia, sickle cell disease, or myelodysplastic syndrome, or may have undergone physical trauma), by administering a composition comprising hepcidin or mini-hepcidin to the subject (e.g., before, during, or after the blood transfusion).

In some aspects, provided herein are methods for reducing total body iron in a subject having acquired iron overload (e.g., iron overload resulting from a cardiovascular surgery, cardiopulmonary bypass, acute coronary syndrome, or sepsis) by administering a composition comprising hepcidin or mini-hepcidin to the subject according to any of the methods discussed herein. In some such embodiments, the subject is undergoing cardiovascular surgery such as a cardiopulmonary bypass. In other such embodiments, the subject has previously undergone cardiovascular surgery such as a cardiopulmonary bypass.

In some aspects, provided herein are methods of reducing total body iron in a subject by administering hepcidin or mini-hepcidin, wherein the subject has a condition, for example, insulin resistance and insufficiency (diabetes), carotid artery lesion, chronic kidney disease, acute kidney injury, proteinuria, anti-glomerular basement membrane (anti-GMB) glomerulonephritis, minimal change disease (nephrotic syndrome), membrane nephropathy, or autoimmune glomerulonephritis (e.g., immune complex induced glomerulonephritis). In certain embodiments, the condition is caused by acquired iron overload.

In further aspects, provided herein are methods of reducing total body iron in a subject by administering hepcidin or mini-hepcidin in combination with an iron chelation therapy and/or an iron-deficient diet. Certain embodiments provide methods of reducing total body iron in a subject by administering hepcidin or mini-hepcidin instead of (i.e., in the absence of) an iron chelation therapy and/or an iron-deficient diet. Further embodiments provide methods of reducing total body iron in a subject by administering hepcidin or mini-hepcidin as the only therapy administered to treat and/or prevent iron overload.

In even further embodiments of the invention, an iron chelation therapy and/or an iron-deficient diet administered to a subject to treat and/or prevent iron overload is replaced (e.g., by discontinuing the iron chelation therapy and/or iron-deficient diet) with administering hepcidin or mini-hepcidin to the subject. In certain such embodiments, the iron chelation therapy and/or the iron-deficient diet administered to the subject can be discontinued and after, for example, one day, two days, three days, four days, five days, six days, seven days, eight days, nine days, ten days, eleven days, twelve days, thirteen days, or fourteen days, hepcidin or mini-hepcidin begins to be administered to the subject. In other such embodiments, administering hepcidin or mini-hepcidin to the subject who is receiving the iron chelation therapy and/or an iron-deficient diet is commenced and after, for example, one day, two days, three days, four days, five days, six days, seven days, eight days, nine days, ten days, eleven days, twelve days, thirteen days, or fourteen days, the iron chelation therapy and/or the iron-deficient diet administered to the subject is discontinued.

### I. Dosing

The method may comprise administering about 10 μg to about 1 gram of hepcidin or mini-hepcidin to the subject, such as about 100 μg to about 100 mg, about 200 μg to about 50 mg, or about 500 μg to about 10 mg, about 500 μg to about 5 mg, or about 500 μg to about 2 mg of hepcidin or mini-hepcidin. The method may comprise administering about 100 μg, about 150 μg, about 200 μg, about 250 μg, about 300 μg, about 333 μg, about 400 μg, about 500 μg, about 600 μg, about 667 μg, about 700 μg, about 750 μg, about 800 μg, about 850 μg, about 900 μg, about 950 μg, about 1000 μg, about 1200 μg, about 1250 μg, about 1300 μg, about 1333 μg, about 1350 μg, about 1400 μg, about 1500 μg, about 1667 μg, about 1750 μg, about 1800 μg, about 2000 μg, about 2200 μg, about 2250 μg, about 2300 μg, about 2333 μg, about 2350 μg, about 2400 μg, about 2500 μg, about 2667 μg, about 2750 μg, about 2800 μg, about 3 mg, about 3.3 mg, about 3.5 mg, about 3.7 mg, about 4 mg, about 4.5 mg, about 5 mg, about 6 mg, about 7 mg, about 8 mg, about 9 mg, or about 10 mg of hepcidin or mini-hepcidin.

Administering a composition comprising hepcidin or mini-hepcidin to the subject may comprise administering a bolus of the composition.

The method may comprise administering the composition to the subject at least once per month, such as at least once per week. The method may comprise administering the composition to the subject 1, 2, 3, 4, 5, 6, or 7 times per week. In preferred embodiments, the method comprises administering the composition to the subject 1, 2, or 3 times per week.

The method may comprise administering about 10 μg to about 1 gram of hepcidin or mini-hepcidin to the subject each time the composition is administered, such as about 100 μg to about 100 mg, about 200 μg to about 50 mg, about 500 μg to about 10 mg, about 500 μg to about 5 mg, or about 500 μg to about 2 mg of hepcidin or mini-hepcidin. The method may comprise administering about 100 μg, about 150 μg, about 200 μg, about 250 μg, about 300 μg, about 333 μg, about 400 μg, about 500 μg, about 600 μg, about 667 μg, about 700 μg, about 750 μg, about 800 μg, about 850 μg, about 900 μg, about 950 μg, about 1000 μg, about 1200 μg, about 1250 μg, about 1300 μg, about 1333 μg, about 1350 μg, about 1400 μg, about 1500 μg, about 1667 μg, about 1750 μg, about 1800 μg, about 2000 μg, about 2200 μg, about 2250 μg, about 2300 μg, about 2333 μg, about 2350 μg, about 2400 μg, about 2500 μg, about 2667 μg, about 2750 μg, about 2800 μg, about 3 mg, about 3.3 mg, about 3.5 mg, about 3.7 mg, about 4 mg, about 4.5 mg, about 5 mg, about 6 mg, about 7 mg, about 8 mg, about 9 mg, or about 10 mg of hepcidin or mini-hepcidin to the subject each time the composition is administered.

In some embodiments, less than about 200 mg hepcidin or mini-hepcidin is administered to a human subject each time the composition is administered. In some embodiments, less than about 150 mg hepcidin or mini-hepcidin is administered to a human subject each time the composition is administered, such as less than about 100 mg, less than about 90 mg, less than about 80 mg, less than about 70 mg, less than about 60 mg, or less than about 50 mg.

In some embodiments, less than 10 mg of hepcidin or mini-hepcidin is administered to a human subject each time the composition is administered, such as less than about 9 mg, less than about 8 mg, less than about 7 mg, less than about 6 mg, less than about 5 mg, less than about 4 mg, less than about 3 mg, less than about 2 mg, or less than about 1 mg. In some embodiments, about 100 μg to about 10 mg of hepcidin or mini-hepcidin is administered to a human subject each time the composition is administered, such as about 100 μg to about 9 mg, about 100 μg to about 8 mg, about 100 μg to about 7 mg, about 100 μg to about 6 mg, about 100 μg to about 5 mg, about 100 μg to about 4 mg, about 100 μg to about 3 mg, about 100 μg to about 2 mg, or about 100 μg to about 1 mg.

### II. Indications

In certain aspects, provided herein are methods of treating and/or preventing iron overload in a subject who has acquired iron overload. In some aspects, provided herein are methods for treating and/or preventing a condition (e.g., iron overload resulting from cardiovascular surgery such as a cardiopulmonary bypass, acute coronary syndrome or sepsis) in a subject by administering a composition comprising hepcidin or mini-hepcidin to the subject. In some embodiments, the condition is comorbid with iron overload (e.g., acquired iron overload or non-acquired iron overload). In some embodiments, the subject is undergoing a cardiovascular surgery such as cardiopulmonary bypass. In some embodiments, the subject has previously undergone cardiovascular surgery such as a cardiopulmonary bypass.

In some embodiments, the subject has undergone a blood transfusion or cardiovascular surgery such as a cardiopulmonary bypass (e.g., within the past day, 2 days, 3 days, 4 days, 5 days, 6 days, week, 2 weeks, 3 weeks, 4 weeks, month, 2 months, 3 months, 4 months, 5 months, 6 months). In some embodiments, the subject has undergone at least 1, at least 2, at least 3, at least 4 or at least 5 blood transfusions within the past week. In some embodiments, the subject has undergone at least 1, at least 2, at least 3, at least 4, at least 5, at least 6, at least 7, at least 8, at least 9 or at least 10 blood transfusions within the past month. In some embodiments, the subject has undergone at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19 or at least 20 blood transfusions within the past six months. In some embodiments, the subject has undergone at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19 or at least 20 blood transfusions within the past year.

In some embodiments, the subject is a subject who is undergoing a blood transfusion.

In some embodiments, the subject is administered a composition described herein before undergoing a blood transfusion (e.g., no more than 1 day before, no more than 2 days before, no more than 3 days before, no more than 4 days before, no more than 5 days before, no more than 6 days before, or no more than a week before). In some embodiments, the composition is administered at least 1 hour before, at least 2 hours before, at least 3 hours before, at least 4 hours before, at least 5 hours before, at least 6 hours before, at least 7 hours before, at least 8 hours before, at least 9 hours before, at least 10 hours before, at least 11 hours before, at least 12 hours before, at least 13 hours before, at least 14 hours before, at least 15 hours before, at least 16 hours before, at least 17 hours before, at least 18 hours before, at least 19 hours before, at least 20 hours before, at least 21 hours before, at least 22 hours before, at least 23 hours before, or at least 1 day before.

In some embodiments, the subject has a disease or disorder that results in frequent blood transfusions. In some embodiments, the subject has anemia (e.g., aplastic anemia, hemolytic anemia, or sideroblastic anemia). In some embodiments, the subject has thalassemia (e.g., hemoglobin E-beta thalassemia or hemoglobin E thalassemia). In some embodiments, the subject has sickle cell disease. In some embodiments, the subject has myelodysplastic syndrome. In some embodiments, the subject has undergone, is undergoing, or is about to undergo physical trauma. The subject may have a tissue injury (e.g., crush injury or a burn injury). Because kidneys are especially prone to damage resulting from iron overload, in some embodiments, the subject that has undergone, is undergoing, or is about to undergo physical trauma also has a chronic or acute kidney injury.

Disclosed herein are methods for reducing, preventing or reversing organ damage or enhancing organ preservation and/or survival comprising administering compositions provided herein to an organ or to an individual post-mortem, Currently, very few pharmacological agents are known to be effective in organ preservation solutions. Injuries to organs generally increase as a function of the length of time an organ is maintained ex vivo. For example, in the case of a lung, typically it may be preserved ex vim for only about 6 to about 8 hours before it becomes unusable for transplantation. A heart typically may be preserved ex vivo for only about 4 to about 6 hours before it becomes unusable for transplantation. These relatively brief time periods limit the number of recipients who can be reached from a given donor site, thereby restricting the recipient pool for a harvested organ. Even within these time limits, the organs may nevertheless be significantly damaged, even where there may not be any observable indication of the damage. Because of this, sub-optimal organs may be transplanted, resulting in post-transplant organ dysfunction or other injuries, Thus, it would be desirable to develop techniques that can reduce, prevent or reverse organ damage thereby extending the time during which an organ can be preserved in a healthy state ex vivo. Such techniques would reduce the risk of post-transplant organ failure.

In some aspects, provided herein are methods and compositions to prevent organ or tissue damage to an organ (e.g., an organ for transplant) or an organ donor. For example, an organ, or organ donor, may be perfused post-mortem with compositions provided herein to prevent damage to the organ. Also provided herein are methods for reducing, preventing or reversing organ damage or enhancing organ preservation and/or survival comprising administering a composition disclosed herein. In certain embodiments, the composition is administered to the organ and/or organ donor less than 24 hours prior to removal of the organ, such as less than 12, eight, six, four or two hours prior to removal of the organ. In certain embodiments, the composition is administered to the organ and/or organ donor immediately prior to removal of the organ (e.g., less than one hour prior to removal of the organ, such as less than 30, 15, or 10 minutes prior to removal of the organ). In certain embodiments, the organ donor is a human.

In some embodiments, provided herein are methods of facilitating an organ transplant procedure and/or enhancing the success of an organ transplant procedure, including bone marrow transplant, comprising administering a composition disclosed herein i.e., a composition comprising hepcidin or mini-hepcidin) to the organ or organ donor prior to transplantation. In some aspects, provided herein are methods and compositions for prolonging organ viability ex vivo, comprising administering a compound disclosed herein (i.e., a composition comprising hepcidin or mini-hepcidin). In certain embodiments, the organ is contacted with a composition disclosed herein while the organ is still in a body, during the removal of the organ from a body, after the organ is removed from a body, while the organ is being transplanted into a recipient, immediately after the organ is transplanted into a recipient, or any combination thereof.

In some embodiments, the organ in contact with, and preferably partially or wholly submersed in, an organ preservation solution, wherein the organ preservation solution comprises a composition disclosed herein. In certain embodiments, the organ preservation solution further comprises potassium, sodium, magnesium, calcium, phosphate, sulphate, glucose, citrate, mannitol, histidine, tryptophan, alpha-ketoglutaric acid, lactobionate, raffinose, adenosine, allopurinol, glutathione, glutamate, insulin, dexamethasone, hydroxyethyl starch, bactrim, trehalose, gluconate, or combinations thereof. In certain embodiments, the organ preservation solution comprises sodium, potassium, magnesium, or combinations thereof. In certain embodiments, the organ preservation solution is free or substantially free of cells, coagulation factors, DNA, and/or plasma proteins. In certain embodiments, the organ preservation solution is sterile.

In further aspects, provided herein are methods of treating and/or preventing a condition, for example, insulin resistance, insulin insufficiency (diabetes), carotid artery lesions, chronic kidney disease, acute kidney injury, proteinuria, anti-glomerular basement membrane (anti-GMB) glomerulonephritis, minimal change disease (nephrotic syndrome), membrane nephropathy, autoimmune glomerulonephritis (e.g., immune complex induced glomerulonephritis), conditions associated with reduced iron absorption by bone marrow (e.g., conditions where the bone marrow is compromised, such as conditions in which compromised bone marrow leads to acute increase in serum iron because iron no longer being consumed by the bone marrow), by administering a composition comprising hepcidin or mini-hepcidin to a subject. In certain such embodiments, the condition is caused or exacerbated by acquired iron overload in the subject. In some embodiments, an individual has total body iron within normal or average physiological ranges (e.g., the subject may have transient iron overload or no iron overload). In some embodiments, an individual has a level of total body iron above normal or average physiological ranges.

Increases in dietary iron content, a modest elevation of total body iron, or an increase of iron in localized areas of the body are associated with insulin resistance and disorders associated with insulin resistance (e.g., metabolic syndrome). In some embodiments, provided herein are methods of treating and/or preventing insulin resistance and insulin insufficiency (e.g., diabetes) by administering a composition comprising hepcidin or mini-hepcidin to a subject. Additionally, iron overload can cause apoptosis of beta cells, which are susceptible to oxidative stress due to their limited antioxidant capacity and high affinity for iron uptake. Therefore, provided herein are methods of reducing apoptosis of beta cells in a patient (e.g., a patient with diabetes (e.g., type 2 diabetes) and/or insulin resistance). In some embodiments, an individual has total body iron within normal or average physiological ranges (e.g., the subject may have transient iron overload or no iron overload). In some embodiments, an individual has a level of total body iron above normal or average physiological ranges. Carotid artery lesions in humans contain large amounts of iron. In patients with carotid atherosclerosis, serum ferritin level correlates with the level of low molecular weight iron compounds and lipid peroxidation products in the carotid endarterectomy specimens. The interaction of iron and lipoproteins in plaque promotes plaque instability by inducing foam cell apoptosis. In some embodiments, provided herein are methods of treating carotid artery lesion by administering a composition comprising hepcidin or mini-hepcidin to a subject. In some embodiments, provided herein are methods of reducing the amount of iron in a carotid artery lesion by administering a composition comprising hepcidin or mini-hepcidin to a subject.

Iron can accumulate in the renal tissue in various models of acute kidney injury, and iron chelation therapy attenuates renal injury and dysfunction. Proteinuria results in accumulation of iron in the proximal tubular epithelial cells, subsequently causing cell damage. Iron chelation therapy or an iron deficient diet ameliorate proteinuria and improve renal function and structure in animal models of anti-GBM glomerulonephritis, puromycin-induced minimal change disease, membranous nephropathy and immune complex glomerulonephritis. Therefore, provided herein are methods of treating chronic kidney disease, acute kidney injury, proteinuria, anti-glomerular basement membrane (anti-GMB) glomerulonephritis, minimal change disease (nephrotic syndrome), membrane nephropathy, or autoimmune glomerulonephritis (e.g., immune complex induced glomerulonephritis) by administering a composition comprising hepcidin or mini-hepcidin to a subject.

Iron overload increases the risk of infections in patients with chronic kidney disease. Therefore, provided herein are methods of reducing the risk of infection in patients with chronic kidney disease by administering a composition comprising hepcidin or mini-hepcidin to a subject. In some embodiments, the patient is undergoing dialysis.

The methods disclosed herein may comprise conjoint administration of a composition comprising hepcidin or mini-hepcidin and any chelator or chelation therapy.

### III. Subjects

The subject may be a mammal. The subject may be a rodent, lagomorph, feline, canine, porcine, ovine, bovine, equine, or primate. In preferred embodiments, the subject is a human. The subject may be a female or male. The subject may be an infant, child, or adult.

In some embodiments, the serum iron concentration of the subject is at least about 50 μg/dL prior to administering the composition, such as at least about 55 μg/dL, at least about 60 μg/dL, at least about 65 μg/dL, at least about 70 μg/dL, at least about 75 μg/dL, at least about 80 μg/dL, at least about 85 μg/dL, at least about 90 μg/dL, at least about 95 μg/dL, at least about 100 μg/dL, at least about 110 μg/dL, at least about 120 μg/dL, at least about 130 μg/dL, at least about 140 μg/dL, at least about 150 μg/dL, at least about 160 μg/dL, at least about 170 μg/dL, at least about 175 μg/dL, at least about 176 μg/dL, at least about 177 μg/dL, at least about 180 μg/dL, at least about 190 μg/dL, at least about 200 μg/dL, at least about 210 μg/dL, at least about 220 μg/dL, at least about 230 μg/dL, at least about 240 μg/dL, at least about 250 μg/dL, at least about 260 μg/dL, at least about 270 μg/dL, at least about 280 μg/dL, at least about 290 μg/dL, or at least about 300 μg/dL. The serum iron concentration of the subject may be about 50 μg/dL to about 500 μg/dL prior to administering the composition, such as about 55 μg/dL to about 500 μg/dL, about 60 μg/dL to about 500 μg/dL, about 65 μg/dL to about 500 μg/dL, about 70 μg/dL to about 500 μg/dL, about 75 μg/dL to about 500 μg/dL, about 80 μg/dL to about 500 μg/dL, about 85 μg/dL to about 500 μg/dL, about 90 μg/dL to about 500 μg/dL, about 95 μg/dL to about 500 μg/dL, about 100 μg/dL to about 500 μg/dL, about 110 μg/dL to about 500 μg/dL, about 120 μg/dL to about 500 μg/dL, about 130 μg/dL to about 500 μg/dL, about 140 μg/dL to about 500 μg/dL, about 150 μg/dL to about 500 μg/dL, about 160 μg/dL to about 500 μg/dL, about 170 μg/dL to about 500 μg/dL, about 175 μg/dL to about 500 μg/dL, about 176 μg/dL to about 500 μg/dL, about 177 μg/dL to about 500 μg/dL, about 180 μg/dL to about 500 μg/dL, about 190 μg/dL to about 500 μg/dL, about 200 μg/dL to about 500 μg/dL, about 210 μg/dL to about 500 μg/dL, about 220 μg/dL to about 500 μg/dL, about 230 μg/dL to about 500 μg/dL, about 240 μg/dL to about 500 μg/dL, about 250 μg/dL to about 500 μg/dL, about 260 μg/dL to about 500 μg/dL, about 270 μg/dL to about 500 μg/dL, about 280 μg/dL to about 500 μg/dL, about 290 μg/dL to about 500 μg/dL, or about 300 μg/dL to about 500 μg/dL.

In preferred embodiments, administering the composition to a subject decreases the serum iron concentration of the subject. For example, administering the composition may decrease the serum iron concentration of a subject by at least about 5 μg/dL, at least about 10 μg/dL, at least about 5 μg/dL, at least about 20 μg/dL, at least about 30 μg/dL, at least about 40 μg/dL, at least about 50 μg/dL, at least about 60 μg/dL, at least about 70 μg/dL, at least about 80 μg/dL, at least about 90 μg/dL, or at least about 100 μg/dL. Administering the composition may decrease the serum iron concentration of the subject for at least 24 hours. For example, administering the composition may decrease the serum iron concentration of the subject by at least about 5 μg/dL for a period of time of at least 24 hours. Administering the composition may decrease the serum iron concentration of the subject by at least about 5 μg/dL for at least 4 hours, at least 6 hours, or at least 12 hours. Administering the composition may decrease the serum iron concentration of the subject by at least about 5 μg/dL for at least 1 day, at least 2 days, at least 3 days, at least 4 days, at least 5 days, at least 6 days, at least 7 days, or at least 8 days. Administering the composition may decrease the serum iron concentration of the subject by at least about 1%, at least about %, at least about 5%, such as at least about 10%, at least about 15%, at least about 20%, at least about 25%, or at least about 30%. Administering the composition may decrease the serum iron concentration of the subject by at least about 5% for at least 4 hours, at least 6 hours, or at least 12 hours. Administering the composition may decrease the serum iron concentration of the subject by at least about 5% for at least 1 day, at least 2 days, at least 3 days, at least 4 days, at least 5 days, at least 6 days, at least 7 days, or at least 8 days.

In some embodiments, the subject has a serum hepcidin concentration of less than about 1000 ng/mL prior to administering the composition, such as less than about 900 ng/mL, less than about 800 ng/mL, less than about 700 ng/mL, less than about 600 ng/mL, less than about 500 ng/mL, less than about 400 ng/mL, less than about 300 ng/mL, less than about 200 ng/mL, less than about 100 ng/mL, less than about 90 ng/mL, less than about 80 ng/mL, less than about 70 ng/mL, less than about 60 ng/mL, less than about 50 ng/mL, less than about 40 ng/mL, less than about 30 ng/mL, less than about 20 ng/mL, or less than about 10 ng/mL. The subject may have a serum hepcidin concentration of about 1 ng/mL to about 1000 ng/mL prior to administering the composition, such as about 1 ng/mL to about 900 ng/mL, about 1 ng/mL to about 800 ng/mL, about 1 ng/mL to about 700 ng/mL, about 1 ng/mL to about 600 ng/mL, about 1 ng/mL to about 500 ng/mL, about 1 ng/mL to about 400 ng/mL, about 1 ng/mL to about 300 ng/mL, about 1 ng/mL to about 200 ng/mL, about 1 ng/mL to about 100 ng/mL, about 1 ng/mL to about 90 ng/mL, about 1 ng/mL to about 80 ng/mL, about 1 ng/mL to about 70 ng/mL, about 1 ng/mL to about 60 ng/mL, about 1 ng/mL to about 50 ng/mL, about 1 ng/mL to about 40 ng/mL, about 1 ng/mL to about 30 ng/mL, about 1 ng/mL to about 20 ng/mL, or about 1 ng/mL to about 10 ng/mL.

In some embodiments, the subject has a serum ferritin concentration greater than about 10 ng/mL prior to administering the composition, such as greater than about 20 ng/mL, greater than about 30 ng/mL, greater than about 40 ng/mL, greater than about 50 ng/mL, greater than about 60 ng/mL, greater than about 70 ng/mL, greater than about 80 ng/mL, greater than about 90 ng/mL, greater than about 100 ng/mL, greater than about 200 ng/mL, greater than about 300 ng/mL, greater than about 400 ng/mL, greater than about 500 ng/mL, greater than about 600 ng/mL, greater than about 700 ng/mL, greater than about 800 ng/mL, greater than about 900 ng/mL, greater than about 1000 ng/mL, greater than about 2000 ng/mL, greater than about 3000 ng/mL, greater than about 4000 ng/mL, greater than about 5000 ng/mL, greater than about 6000 ng/mL, greater than about 7000 ng/mL, greater than about 8000 ng/mL, greater than about 9000 ng/mL, or even greater than about 10 μg/mL. The subject may have a serum ferritin concentration of about 10 ng/mL to about 100 μg/mL prior to administering the composition, such as about 20 ng/mL to about 100 μg/mL, about 30 ng/mL to about 100 μg/mL, about 40 ng/mL to about 100 μg/mL, about 50 ng/mL to about 100 μg/mL, about 60 ng/mL to about 100 μg/mL, about 70 ng/mL to about 100 μg/mL, about 80 ng/mL to about 100 μg/mL, about 90 ng/mL to about 100 μg/mL, about 100 ng/mL to about 100 μg/mL, about 200 ng/mL to about 100 μg/mL, about 300 ng/mL to about 100 μg/mL, about 400 ng/mL to about 100 μg/mL, about 500 ng/mL to about 100 μg/mL, about 600 ng/mL to about 100 μg/mL, about 700 ng/mL to about 100 μg/mL, about 800 ng/mL to about 100 μg/mL, about 900 ng/mL to about 100 μg/mL, or about 1000 ng/mL to about 100 μg/mL. The subject may have a serum ferritin concentration of about 10 ng/mL to about 20 μg/mL prior to administering the composition, such as about 20 ng/mL to about 20 μg/mL, about 30 ng/mL to about 20 μg/mL, about 40 ng/mL to about 20 μg/mL, about 50 ng/mL to about 20 μg/mL, about 60 ng/mL to about 20 μg/mL, about 70 ng/mL to about 20 μg/mL, about 80 ng/mL to about 20 μg/mL, about 90 ng/mL to about 20 μg/mL, about 100 ng/mL to about 20 μg/mL, about 200 ng/mL to about 20 μg/mL, about 300 ng/mL to about 20 μg/mL, about 400 ng/mL to about 20 μg/mL, about 500 ng/mL to about 20 μg/mL, about 600 ng/mL to about 20 μg/mL, about 700 ng/mL to about 20 μg/mL, about 800 ng/mL to about 20 μg/mL, about 900 ng/mL to about 20 μg/mL, or about 1000 ng/mL to about 20 μg/mL.

In some embodiments, the subject has a serum ferritin concentration of less than about 10 μg /mL prior to administering the composition, such as less than about 1000 ng/mL, less than about 900 ng/mL, less than about 800 ng/mL, less than about 700 ng/mL, less than about 600 ng/mL, less than about 500 ng/mL, less than about 400 ng/mL, less than about 300 ng/mL, less than about 200 ng/mL, less than about 100 ng/mL, less than about 90 ng/mL, less than about 80 ng/mL, less than about 70 ng/mL, less than about 60 ng/mL, less than about 50 ng/mL, less than about 40 ng/mL, less than about 30 ng/mL, less than about 20 ng/mL, or less than about 10 ng/mL. The subject may have a serum ferritin concentration of about 1 ng/mL to about 1000 ng/mL prior to administering the composition, such as about 1 ng/mL to about 900 ng/mL, about 1 ng/mL to about 800 ng/mL, about 1 ng/mL to about 700 ng/mL, about 1 ng/mL to about 600 ng/mL, about 1 ng/mL to about 500 ng/mL, about 1 ng/mL to about 400 ng/mL, about 1 ng/mL to about 300 ng/mL, about 1 ng/mL to about 200 ng/mL, about 1 ng/mL to about 100 ng/mL, about 1 ng/mL to about 90 ng/mL, about 1 ng/mL to about 80 ng/mL, about 1 ng/mL to about 70 ng/mL, about 1 ng/mL to about 60 ng/mL, about 1 ng/mL to about 50 ng/mL, about 1 ng/mL to about 40 ng/mL, about 1 ng/mL to about 30 ng/mL, about 1 ng/mL to about 20 ng/mL, or about 1 ng/mL to about 10 ng/mL.

In some embodiments, administering the composition decreases the serum ferritin concentration of the subject. For example, administering the composition may decrease the serum ferritin concentration of the subject by at least about 10 ng/mL, at least about 20 ng/mL, at least about 30 ng/mL, at least about 40 ng/mL, at least about 50 ng/mL, at least about 60 ng/mL, at least about 70 ng/mL, at least about 80 ng/mL, at least about 90 ng/mL, or at least about 100 ng/mL.

In some embodiments, the subject has a total body iron content of about 40 to about 50 mg/kg prior to administering the composition. The subject may have a total body iron content greater than about 50 mg/kg prior to administering the composition, such as greater than about 55 mg/kg, greater than about 60 mg/kg, greater than about 65 mg/kg, or greater than about 70 mg/kg.

In some embodiments, the subject has a transferrin saturation percentage greater than about 10% prior to administering the composition, such as greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, or even greater than about 90%. The subject may have a transferrin saturation percentage of about 10% to about 99% prior to administering the composition, such as about 15% to about 99%, about 20% to about 99%, about 25% to about 99%, about 30% to about 99%, about 35% to about 99%, about 40% to about 99%, about 45% to about 99%, about 50% to about 99%, about 55% to about 99%, about 60% to about 99%, about 65% to about 99%, about 70% to about 99%, about 75% to about 99%, about 80% to about 99%, or about 85% to about 99%. The subject may have a transferrin saturation percentage of about 10% to about 95% prior to administering the composition, such as about 15% to about 95%, about 20% to about 95%, about 25% to about 95%, about 30% to about 95%, about 35% to about 95%, about 40% to about 95%, about 45% to about 95%, about 50% to about 95%, about 55% to about 95%, about 60% to about 95%, about 65% to about 95%, about 70% to about 95%, about 75% to about 95%, about 80% to about 95%, or about 85% to about 95%.

In some embodiments, administering the composition decreases the transferrin saturation percentage of the subject. For example, administering the composition to a subject may decrease the transferrin saturation percentage of the subject by at least about 1% transferrin saturation, such as at least about 2% transferrin saturation, at least about 3% transferrin saturation, at least about 4% transferrin saturation, at least about 5% transferrin saturation, at least about 6% transferrin saturation, at least about 7% transferrin saturation, at least about 8% transferrin saturation, at least about 9% transferrin saturation, at least about 10% transferrin saturation, at least about 11% transferrin saturation, at least about 12% transferrin saturation, at least about 13% transferrin saturation, at least about 14% transferrin saturation, at least about 15% transferrin saturation, at least about 16% transferrin saturation, at least about 17% transferrin saturation, at least about 18% transferrin saturation, at least about 19% transferrin saturation, at least about 20% transferrin saturation, at least about 25% transferrin saturation, at least about 30% transferrin saturation, at least about 35% transferrin saturation, at least about 40% transferrin saturation, at least about 45% transferrin saturation, or at least about 50% transferrin saturation.

### IV. Active Agent

The hepcidin peptide is a 25-amino acid peptide with the amino acid sequence set forth in SEQ ID NO:1. The hepcidin peptide is a cleavage product of a larger protein, and the cell membrane protein furin can convert an extracellular hepcidin precursor protein into the hepcidin peptide. The term “hepcidin” as used herein may therefore refer to a peptide comprising the sequence set forth in SEQ ID NO:1, including peptides that are longer than 25 amino acids, such as peptides consisting of 26 to 100 amino acids. Conservative amino acid substitutions, additions, and deletions may be made to SEQ ID NO:1 without significantly affecting the function of hepcidin. Thus, the term “hepcidin” may refer to a peptide comprising an amino acid sequence having at least 90%, 91%, 92%, 93%, 94%, 95%, or 96% sequence homology with the amino acid sequence set forth in SEQ ID NO:1. Sequence homology may be determined using any suitable sequence alignment program, such as Protein Blast (blastp) or Clustal (e.g., Clustal, ClustalW, ClustalX, or Clustal Omega), e.g., using default parameters, such as default weights for gap openings and gap extensions. Sequence homology may refer to sequence identity. The term “hepcidin” may refer to a peptide comprising an amino acid sequence that is identical to the sequence set forth in SEQ ID NO:1 except that 1, 2, 3, 4, 5, 6, 7, 8, 9, or 10 amino acids of SEQ ID NO:1 are substituted with different amino acids. In preferred embodiments, hepcidin comprises a cysteine at each of the positions in which a cysteine occurs in SEQ ID NO:1.

SEQ ID NO: 1
DTHFPICIFCCGCCHRSKCGMCCKT

N-terminal and C-terminal residues may be deleted from the hepcidin peptide without significantly affecting its function. Thus, in some embodiments, hepcidin refers to a peptide comprising the sequence set forth in SEQ ID NO:2, SEQ ID NO:3, or SEQ ID NO:4, or a peptide comprising an amino acid sequence having at least 90%, 91%, 92%, 93%, 94%, 95%, or 96% sequence homology with the amino acid sequence set forth in SEQ ID NO:2, SEQ ID NO:3, SEQ ID NO:4, or SEQ ID NO:5. The term hepcidin may refer to a peptide comprising an amino acid sequence that is identical to the sequence set forth in SEQ ID NO:2, SEQ ID NO:3, SEQ ID NO:4, or SEQ ID NO:5 except that 1, 2, 3, 4, 5, 6, 7, 8, 9, or 10 amino acids of SEQ ID NO:2, SEQ ID NO:3, SEQ ID NO:4, or SEQ ID NO:5 are substituted with different amino acids. In preferred embodiments, hepcidin comprises a cysteine at each of the positions in which a cysteine occurs in SEQ ID NO:2, SEQ ID NO:3, SEQ ID NO:4, or SEQ ID NO:5.

SEQ ID NO: 2
PICIFCCGCCHRSKCGMCCKT
SEQ ID NO: 3
PICIFCCGCCHRSKCGMCC
SEQ ID NO: 4
ICIFCCGCCHRSKCGMCCKT
SEQ ID NO: 5
CIFCCGCCHRSKCGMCC

In some embodiments, the term “hepcidin” refers to a peptide comprising an amino acid sequence that is identical to the sequence set forth in SEQ ID NO:6, SEQ ID NO:7, SEQ ID NO:8, SEQ ID NO:9, or SEQ ID NO:10. In SEQ ID NO:6, SEQ ID NO:7, SEQ ID NO:8, SEQ ID NO:9, or SEQ ID NO:10, the amino acids labeled “X” may be any amino acid, including naturally occurring and non-naturally occurring amino acids. In some embodiments, each of the amino acids labeled “X” is a naturally occurring amino acid.

SEQ ID NO: 6
XXHXPXCXXCCGCCHRSKCGMCCXX
SEQ ID NO: 7
PXCXXCCGCCHRSKCGMCCKX
SEQ ID NO: 8
PXCXXCCGCCHRSKCGMCC
SEQ ID NO: 9
XCXXCCGCCHRXXCGXCCKX
SEQ ID NO: 10
CXXCCGCCHRXXCGXCC

In preferred embodiments, hepcidin is a molecule that specifically binds to ferroportin and/or iron (e.g., an iron cation). Hepcidin may comprise 1, 2, 3, or 4 disulfide bonds. In preferred embodiments, hepcidin comprises four disulfide bonds. In preferred embodiments, each of the four disulfide bonds is an intramolecular disulfide bond. In preferred embodiments, each of the eight cysteines of SEQ ID NO:1, 2, 3, 4, 5, 6, 7, 8, 9, or 10 participates in one of four intramolecular disulfide bonds with another one of the eight cysteines.

In preferred embodiments, hepcidin has about 10% to 1000% of the activity of a 25 amino acid long peptide comprising the amino acid sequence set forth in SEQ ID NO:1, i.e., wherein the 25 amino acid long peptide comprises the four intramolecular disulfide bonds found in native human hepcidin. For example, hepcidin may have about 50% to about 200% of the activity of a 25 amino acid long peptide comprising the amino acid sequence set forth in SEQ ID NO:1 (i.e., wherein the 25 amino acid long peptide comprises the four intramolecular disulfide bonds found in native human hepcidin), such as about 75% to about 150% of the activity, about 80% to about 120% of the activity, about 90% to about 110% of the activity, or about 95% to about 105% of the activity. The term “activity” may refer to the ability of hepcidin to specifically bind to ferroportin, e.g., thereby inhibiting the transport of intracellular iron into the extracellular space, inhibiting the absorption of dietary iron, and/or reducing serum iron concentration. Activity may refer to the ability of hepcidin to inhibit the transport of intracellular iron into the extracellular space. Activity may refer to the ability of hepcidin to inhibit the absorption of dietary iron. Activity may refer to the ability of hepcidin to reduce serum iron concentration in vivo.

In some embodiments, mini-hepcidin may refer to a mini-hepcidin, modified hepcidin, or a hepcidin mimetic peptide. For the purposes of this application, the terms mini-hepcidin, a modified hepcidin, or a hepcidin mimetic peptide may be used interchangeably. Mini-hepcidins, a modified hepcidin, and hepcidin mimetic peptides are disclosed in US. Pat. Nos. 9,315,545, 9,328,140, and 8,435,941, each of which are hereby incorporated by reference, in particular for their disclosure of compounds that share one or more activities with hepcidin.

A mini-hepcidin may have the structure of Formula I, or a pharmaceutically acceptable salt thereof:

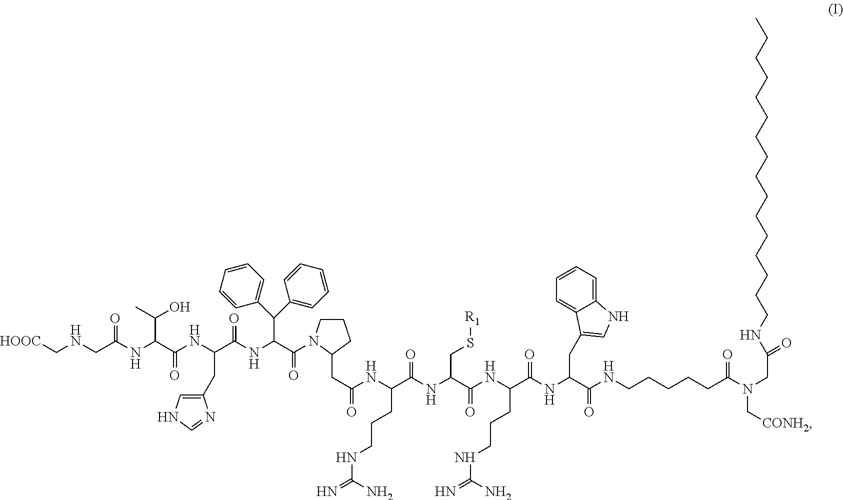

wherein R₁ is, —S-Z₁; -Z₂, —SH, —C(═O)—Z₃ or —S—C(═O)-Z₃,
Z₁ is substituted or unsubstituted C₁-C₁₈ alkyl or C₁-C₁₈ alkenyl, wherein the C₁-C₁₈ alkyl or C₁-C₁₈ alkenyl is branched or unbranched or Z₁ is an electron withdrawing or donating group;
Z₂ is substituted or unsubstituted C₁-C₁₈ alkyl or C₁-C₁₈ alkenyl, wherein the C₁-C₁₈ alkyl or C₁-C₁₈ alkenyl is branched or unbranched or Z₂ is an electron withdrawing or donating group;
Z₃ is substituted or unsubstituted C₁-C₁₈ alkyl or C₁-C₁₈ alkenyl, wherein the C₁-C₁₈ alkyl or C₁-C₁₈ alkenyl is branched or unbranched or Z₃ is an electron withdrawing or donating group.

A mini-hepcidin may have the structure of any one of Formulas II-IV, or a pharmaceutically acceptable salt thereof:

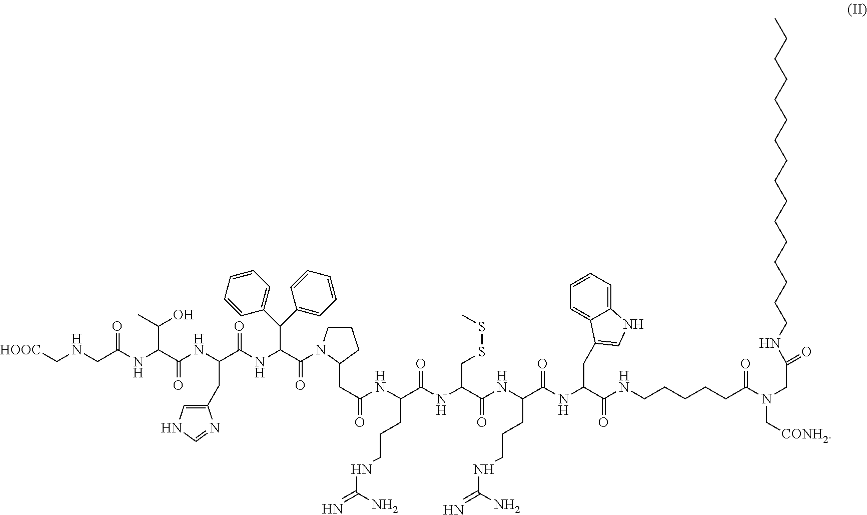 [SMILES: CCCCCCCCCCCCCCCCNC(=O)CN(CC(N)=O)C(=O)CCCCCNC(=O)C(Cc1c[nH]c2ccccc12)NC(=O)C(CCCNC(=N)N)NC(=O)C(CSSC)NC(=O)C(CCCNC(=N)N)NC(=O)CC1CCCN1C(=O)C(NC(=O)C(Cc1c[nH]cn1)NC(=O)C(NC(=O)CNCC(=O)O)C(C)O)C(c1ccccc1)c1ccccc1]

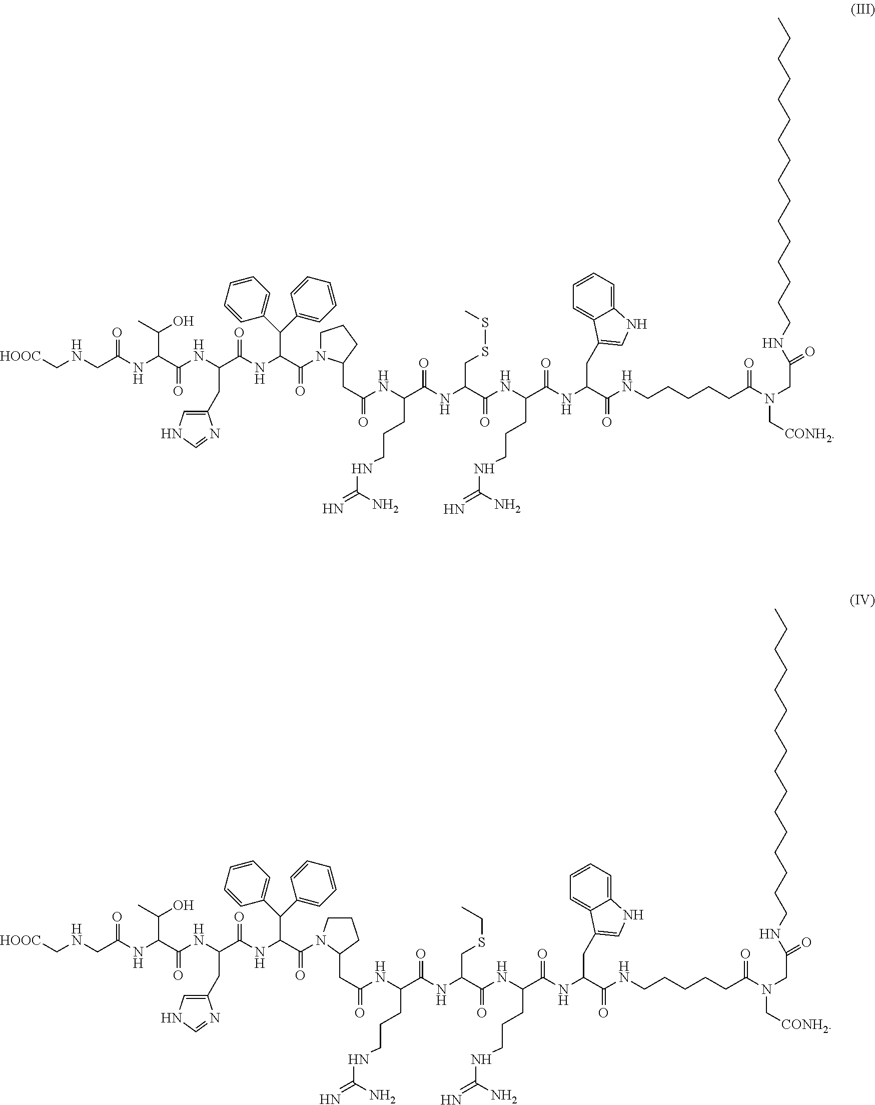 [SMILES: CCCCCCCCCCCCCCCCNC(=O)CN(CC(N)=O)C(=O)CCCCCNC(=O)C(Cc1c[nH]c2ccccc12)NC(=O)C(CCCNC(=N)N)NC(=O)C(CSSC)NC(=O)C(CCCNC(=N)N)NC(=O)CC1CCCN1C(=O)C(NC(=O)C(Cc1c[nH]cn1)NC(=O)C(NC(=O)CNCC(=O)O)C(C)O)C(c1ccccc1)c1ccccc1 ; CCCCCCCCCCCCCCCCNC(=O)CN(CC(N)=O)C(=O)CCCCCNC(=O)C(Cc1c[nH]c2ccccc12)NC(=O)C(CCCNC(=N)N)NC(=O)C(CSCC)NC(=O)C(CCCNC(=N)N)NC(=O)CC1CCCN1C(=O)C(NC(=O)C(Cc1c[nH]cn1)NC(=O)C(NC(=O)CNCC(=O)O)C(C)O)C(c1ccccc1)c1ccccc1]

A mini-hepcidin may have the structure of Formula V, or a pharmaceutically acceptable salt thereof:

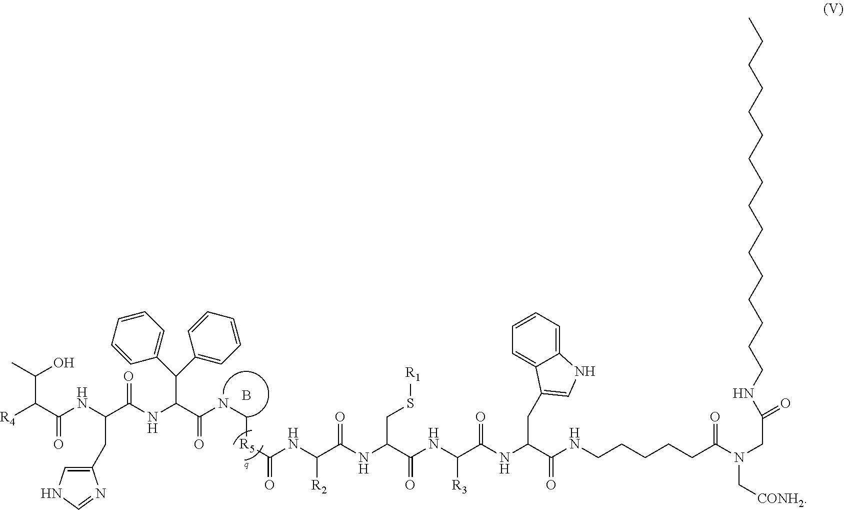

wherein:
R₁ is, H, —S-Z₁; -Z₂, —SH, —C(═O)-Z₃, or —S—C(═O)-Z₃,
R₂ and R₃ are each, independently, optionally substituted C₄-C₇ alkyl,

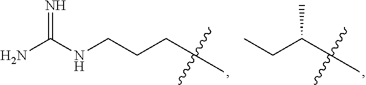

D-Arg, D-Ile, Leu, D-Leu, Thr, D-Thr, Lys, D-Lys, Val, D-Val, D-Nω,ω-dimethyl-arginine, L-Nω,ω-dimethyl-arginine, D-homoarginine, L-homoarginine, D-norarginine, L-norarginine, citrulline, a modified Arg wherein the guanidinium group is modified or substituted, norleucine, norvaline, beta homo-Ile, 1-aminocyclohexane-1-carboxylic acid, N-Me-Arg, N-Me-Ile;
R₄ is Ida, Asp, Acetyl-Asp, (methylamino)pentanedioic acid, Acetyl-Gly-Ida, or Acetyl-Gly-Asp or a derivative thereof to remove its negative charge above pH 4;
R₅ is CR₆R₇, aryl or heteroaryl;
B is absent or forms a 5-7 membered ring; and
q is 0-6, wherein when R₅ aryl or heteroaryl q is 1 and B is absent;
Z₁ is substituted or unsubstituted C₁-C₁₈ alkyl, wherein the C₁-C₁₈ alkyl is branched or unbranched;
Z₂ is substituted or unsubstituted C₁-C₁₈ alkyl, wherein the C₁-C₁₈ alkyl is branched or unbranched;
Z₃ is substituted or unsubstituted C₁-C₁₈ alkyl, wherein the C₁-C₁₈ alkyl is branched or unbranched;
R₆ and R₇ are each, independently, H, halo, optionally substituted C₁-C₃ alkyl, or haloalkyl, provided that when R₁ is H, the compound does not have the structure of Formula XVI.

A mini-hepcidin may have the structure of any one of Formulas VI-VIII, or a pharmaceutically acceptable salt thereof:

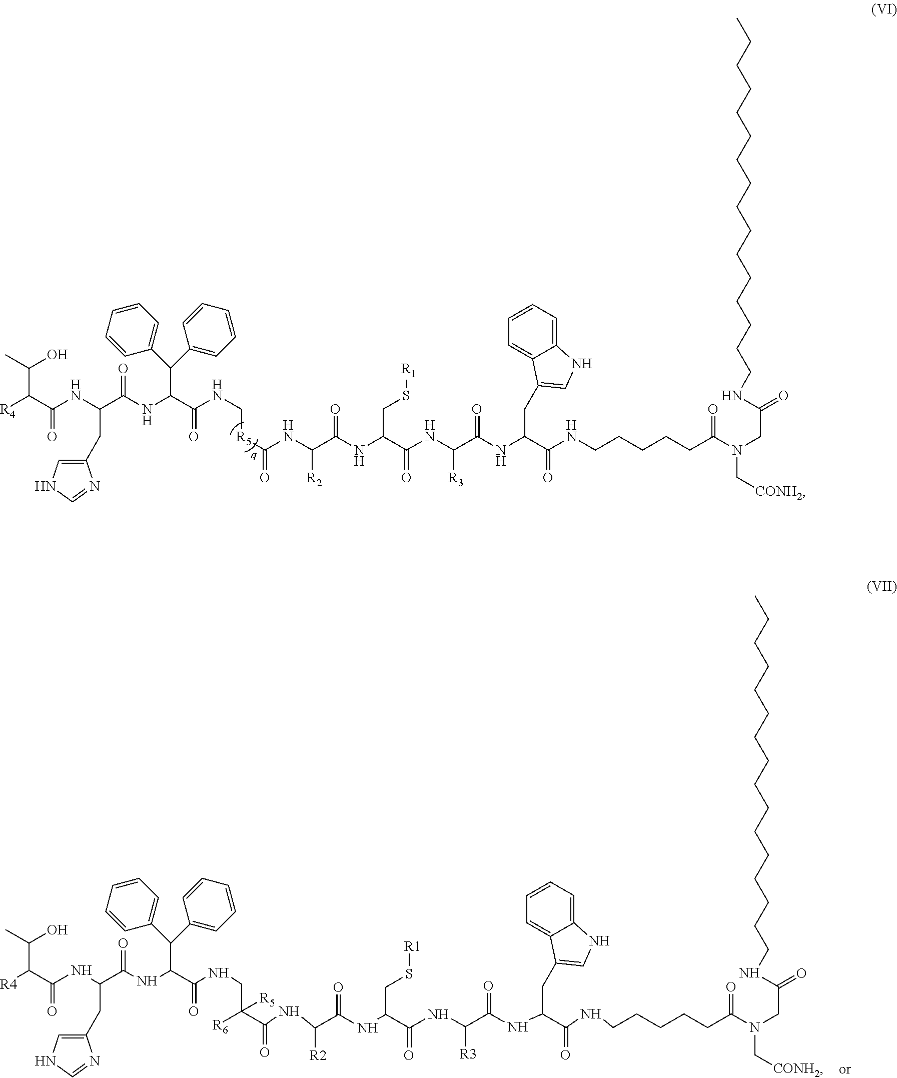

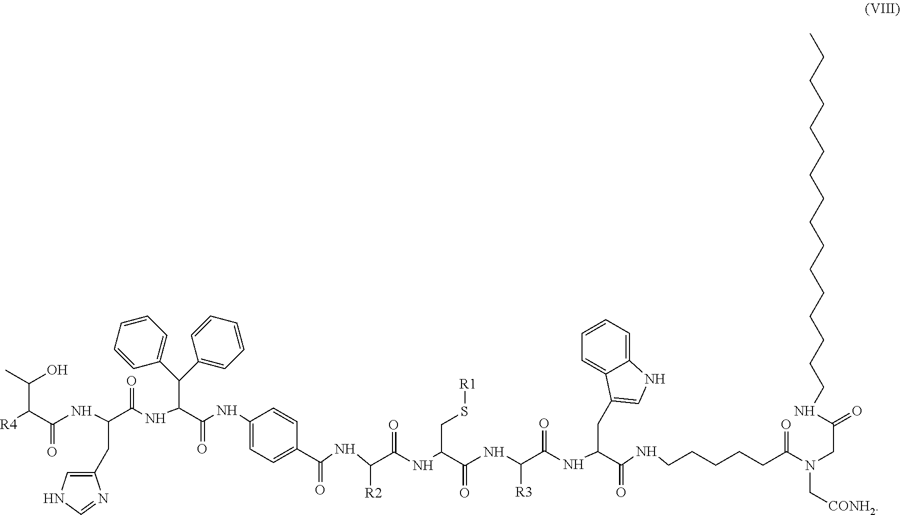

wherein the variables are defined as for Formula V.

A mini-hepcidin may have the structure of Formula IX, or a pharmaceutically acceptable salt thereof:

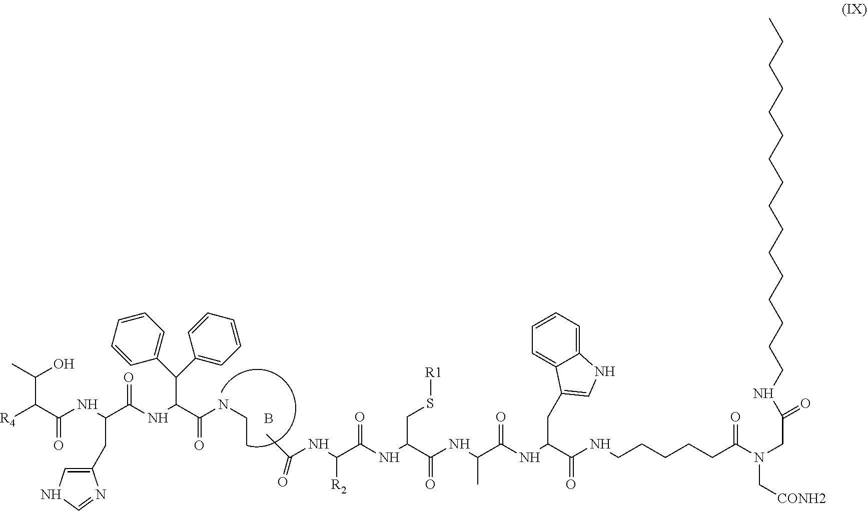

wherein R₁ is H, —S-Z₁, -Z₂, —SH, —S—C(═O)-Z₃, or —C(═O)-Z₃,
R₂ and R₃ are each, independently, optionally substituted C₄-C₇ alkyl,

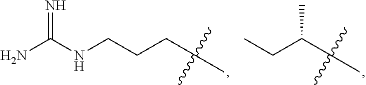

D-Arg, D-Ile, Leu, D-Leu, Thr, D-Thr, Lys, D-Lys, Val, D-Val, D-Nω,ω-dimethyl-arginine, L-Nω,ω-dimethyl-arginine, D-homoarginine, L-homoarginine, D-norarginine, L-norarginine, citrulline, a modified Arg wherein the guanidinium group is modified or substituted, norleucine, norvaline, beta homo-Ile, 1-aminocyclohexane-1-carboxylic acid, N-Me-Arg, N-Me-Ile;
R₄ is Ida, Asp, Acetyl-Asp, (methylamino)pentanedioic acid, Acetyl-Gly-Ida, or Acetyl-Gly-Asp or a derivative thereof to remove its negative charge above pH 4;
B is absent or forms a 5-7 membered ring;
Z₁ is substituted or unsubstituted C₁C₁₈ alkyl, wherein the C₁-C₁₈ alkyl is branched or unbranched;
Z₂ is substituted or unsubstituted C₁-C₁₈ alkyl, wherein the C₁-C₁₈ alkyl is branched or unbranched; and
Z₃ is substituted or unsubstituted C₁-C₁₈ alkyl, wherein the C₁-C₁₈ alkyl is branched or unbranched;
provided that when R₁ is H, the compound does not have the structure of Formula XVI.

A mini-hepcidin may have the structure of Formula X, or a pharmaceutically acceptable salt thereof:

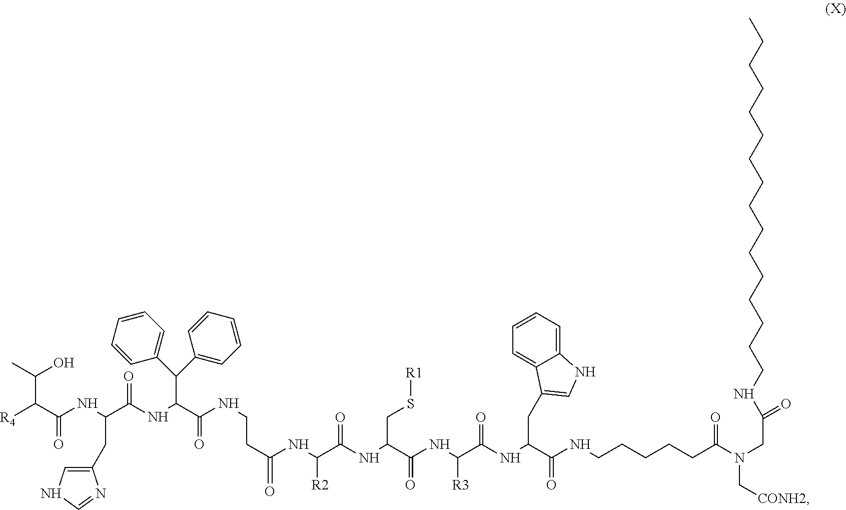

wherein the variables are defined as for Formula IX.

A mini-hepcidin may have the structure of Formula XI, or a pharmaceutically acceptable salt thereof:

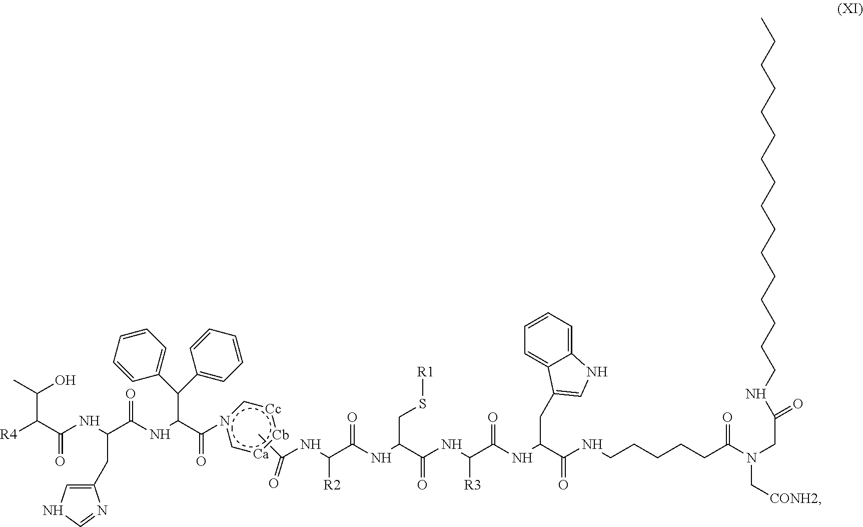

wherein the carbonyl forms a bond with the 6-membered ring at Cₐ, C_{b}, or C_{c} and with the variables as defined for Formula IX.

A mini-hepcidin may have the structure of Formula XII, or a pharmaceutically acceptable salt thereof:

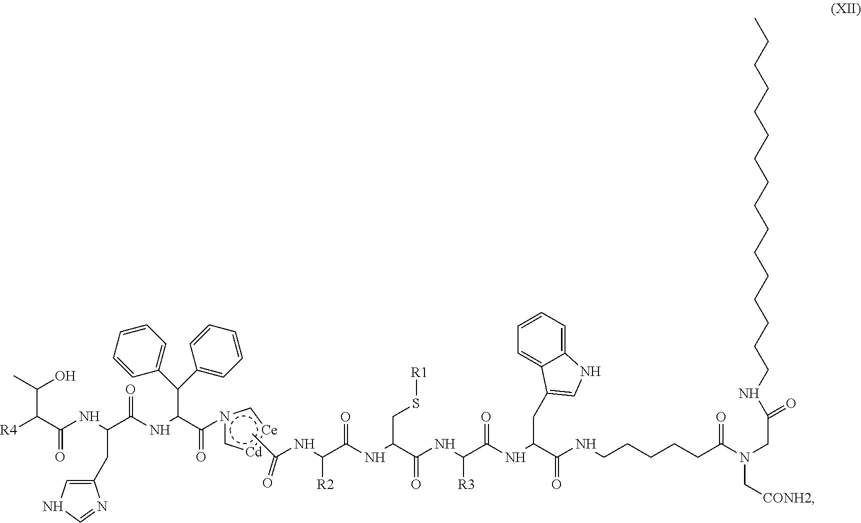

wherein the carbonyl forms a bond with the 5-membered ring at C_{d} or Cₑ. and with the variables as defined for Formula IX.

A mini-hepcidin may have the structure of Formula XIII, or a pharmaceutically acceptable salt thereof:

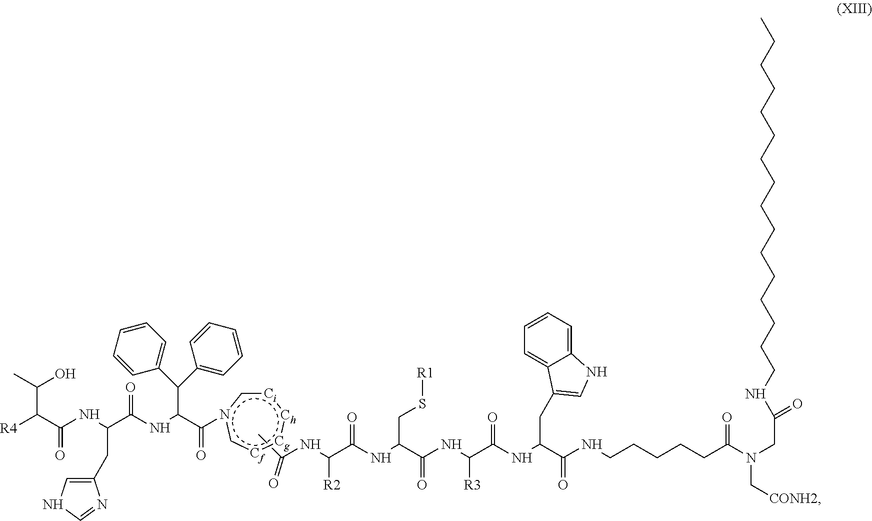

wherein the bond from the carbonyl forms a bond with the 7-membered ring at C_{f}, C_{g}, Cₕ, or Cᵢ and with the variables as defined for Formula IX.

A mini-hepcidin may have the structure of Formula XIV, or a pharmaceutically acceptable salt thereof:

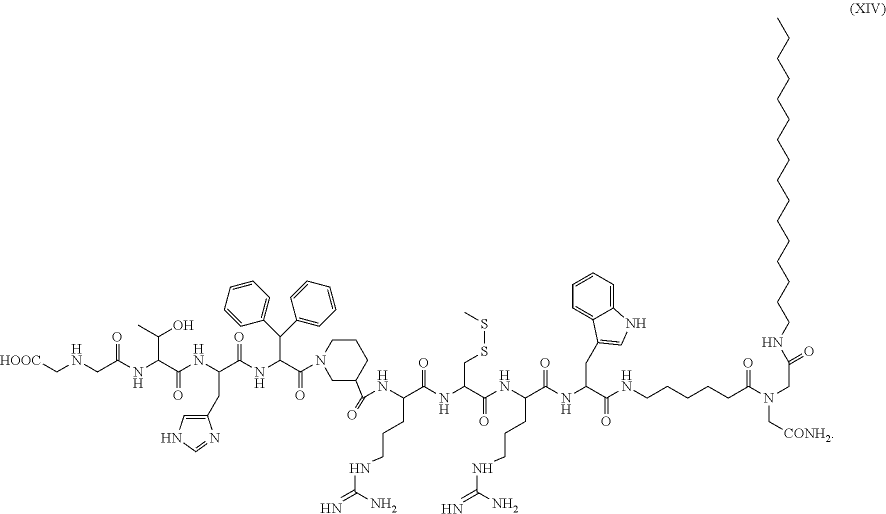 [SMILES: CCCCCCCCCCCCCCCCNC(=O)CN(CC(N)=O)C(=O)CCCCCNC(=O)C(Cc1c[nH]c2ccccc12)NC(=O)C(CCCNC(=N)N)NC(=O)C(CSSC)NC(=O)C(CCCNC(=N)N)NC(=O)C1CCCN(C(=O)C(NC(=O)C(Cc2c[nH]cn2)NC(=O)C(NC(=O)CNCC(=O)O)C(C)O)C(c2ccccc2)c2ccccc2)C1]

A mini-hepcidin may have the structure of Formula XV, or a pharmaceutically acceptable salt thereof:

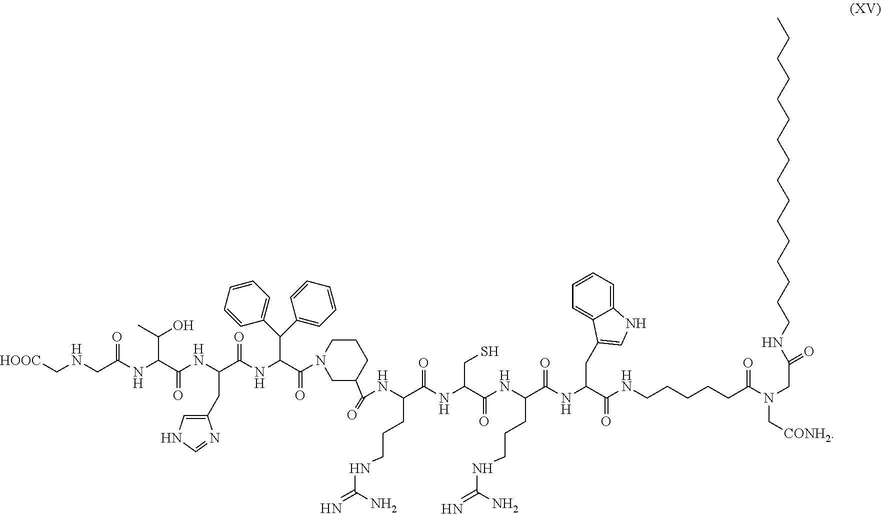 [SMILES: CCCCCCCCCCCCCCCCNC(=O)CN(CC(N)=O)C(=O)CCCCCNC(=O)C(Cc1c[nH]c2ccccc12)NC(=O)C(CCCNC(=N)N)NC(=O)C(CS)NC(=O)C(CCCNC(=N)N)NC(=O)C1CCCN(C(=O)C(NC(=O)C(Cc2c[nH]cn2)NC(=O)C(NC(=O)CNCC(=O)O)C(C)O)C(c2ccccc2)c2ccccc2)C1]

A mini-hepcidin may have the structure of Formula P₁-P₂-P₃-P₄-P₅-P₆-P₇-P₈-P₉-P₁₀ or P₁₀-P₉-P₈-P₇-P₆-P₅-P₄-P₃-P₂-P₁, or a pharmaceutically acceptable salt thereof, wherein P₁ to P₁₀ are as defined in table 1; X₃ is aminohexanoic acid-Ida(NH-PAL)-NH₂, Ida is iminodiacetic acid; Dpa is 3,3-diphenyl-L-alanine; bhPro is beta-homoproline; Npc is L-nipecotic acid; isoNpc is isonipecotic acid; and bAla is beta-alanine.

TABLE 1

| P₁ | P₂ | P₃ | P₄ | P₅ | P₆ | P₇ | P₈ | P₉ | P₁₀ |
|---|---|---|---|---|---|---|---|---|---|
| Ida | Thr | His | Dpa | bhPro | Arg | Cys-S—CH₃ | Arg | Trp | X₃ |
| Ida | Thr | His | Dpa | bhPro | Arg | Cys-C(═O)CH₃ | Arg | Trp | X₃ |
| Ida | Thr | His | Dpa | bhPro | Arg | Cys-CH₂—CH₃ | Arg | Trp | X₃ |
| Ida | Thr | His | Dpa | Npc | Arg | Cys-S—CH₃ | Arg | Trp | X₃ |
| Ida | Thr | His | Dpa | Npc | Arg | Cys | Arg | Trp | X₃ |
| Ida | Thr | His | Dpa | D-Npc | Arg | Cys-S—CH₃ | Arg | Trp | X₃ |
| Ida | Thr | His | Dpa | isoNpc | Arg | Cys-S—CH₃ | Arg | Trp | X₃ |
| Acetyl-Gly-Ida | Thr | His | Dpa | bhPro | Arg | Cys-S—CH₃ | Arg | Trp | X₃ |
| Ida | Thr | His | Dpa | bAla | Arg | Cys-S—CH₃ | Arg | Trp | X₃ |

A mini-hepcidin may have the structure of Formula XVI, or a pharmaceutically acceptable salt thereof:

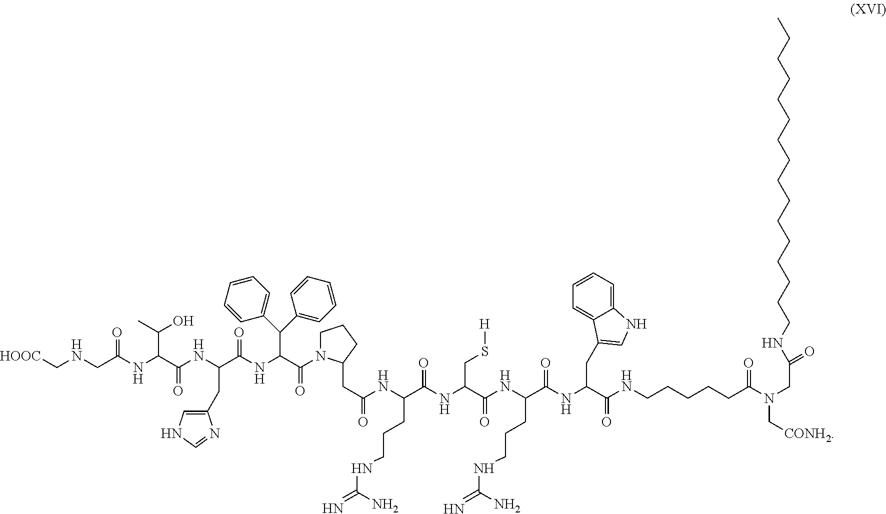 [SMILES: CCCCCCCCCCCCCCCCNC(=O)CN(CC(N)=O)C(=O)CCCCCNC(=O)C(Cc1c[nH]c2ccccc12)NC(=O)C(CCCNC(=N)N)NC(=O)C(CS)NC(=O)C(CCCNC(=N)N)NC(=O)CC1CCCN1C(=O)C(NC(=O)C(Cc1c[nH]cn1)NC(=O)C(NC(=O)CNCC(=O)O)C(C)O)C(c1ccccc1)c1ccccc1]

A mini-hepcidin may have the structure of formula A1-A2-A3-A4-A5-A6-A7-A8-A9-A10, A10-A9-A8-A7-A6-A5-A4-A3-A2-A1, or a pharmaceutically acceptable salt thereof, wherein:
A1 is L-Asp, L-Glu, pyroglutamate, L-Gln, L-Asn, D-Asp, D-Glu, D-pyroglutamate, D-Gln, D-Asn, 3-aminopentanedioic acid, 2,2′-azanediyldiacetic acid, (methylamino)pentanedioic acid, L-Ala, D-Ala, L-Cys, D-Cys, L-Phe, D-Phe, L-Asp, D-Asp, 3,3-diphenyl-L-alanine, 3,3-diphenyl-D-alanine; and if A1 is L-Asp or D-Asp, then A2 is L-Cys or D-Cys; if A1 is L-Phe or D-Phe, then the N-terminus is optionally attached to a PEG molecule linked to chenodeoxvcholate, ursodeoxvcholate, or palmitoyl; or if A1 is 3,3-diphenyl-L-alanine or 3,3-diphenyl-D-alanine, then the N-terminus is attached to palmitoyl;
A2 is L-Thr, L-Ser, L-Val, L-Ala, D-Thr, D-Ser, D-Val, L-tert-leucine, isonipecotic acid, L-α-cyclohexylglycine, bhThr, (2S)-3-hydroxy-2-(methylamino)butanoic acid, D-Ala, L-Cys, D-Cys, L-Pro, D-Pro, or Gly;
A3 is L-His, D-His, 3,3-diphenyl-L-alanine, 3,3-diphenyl-D-alanine, or 2-aminoindane;
A4 is L-Phe, D-Phe, (S)-2-amino-4-phenylbutanoic acid, 3,3-diphenyl-L-alanine, L-biphenylalanine, (1-naphthyl)-L-alanine, (S)-3-Amino-4,4-diphenylbutanoic acid, 4-(aminomethyl)cyclohexane carboxylic acid, (S)-2-amino-3-(perfluorophenyl)propanoic acid, (S)-2-amino-4-phenylbutanoic acid, (S)-2-amino-2-(2,3-dihydro-1H-inden-2-yl)acetic acid, or cyclohexylalanine;
A5 is L-Pro, D-Pro, octahydroindole-2-carboxylic acid, L-β-homoproline, (2S,4S)-4-phenylpyrrolidine-2-carboxylic acid, (2S,5R)-5-phenylpyrrolidine-2-carboxylic acid, or (R)-2-methylindoline;
A6 is L-Ile, D-Ile, L-phenylglycine, L-α-cyclohexylglycine, 4-(aminomethyl)cyclohexane carboxylic acid, (3R)-3-amino-4-methylhexanoic acid, 1-aminocyclohexane-1-carboxylic acid, or (3R)-4-methyl-3-(methylamino)hexanoic acid;
A7 is L-Cys, D-Cys, S-t-Butylthio-L-cysteine, L-homocysteine, L-penicillamine, or D-penicillamine;
A8 is L-Ile, D-Ile, L-α-cyclohexylglycine, 3,3-diphenyl-L-alanine, (3R)-3-amino-4-methylhexanoic acid, 1-aminocyclohexane-1-carboxylic acid, or (3R)-4-methyl-3-(methylamino)hexanoic acid;
A9 is L-Phe, L-Leu, L-Ile, L-Tyr, D-Phe, D-Leu, D-Ile, (S)-2-amino-3-(perfluorophenyl)propanoic acid, N-methyl-phenylalainine, benzylamide, (S)-2-amino-4-phenylbutanoic acid, 3,3-diphenyl-L-alanine, L-biphenylalanine, (1-naphthyl)-L-alanine, (S)-3-amino-4,4-diphenylbutanoic acid, cyclohexylalanine, L-Asp, D-Asp, or cysteamide, wherein L-Phe or D-Phe are optionally linked at the N-terminus to RA, wherein RA is —CONH-CH₂—CH₂—S—, or D-Pro linked to Pro-Lys or Pro-Arg, or L-β-homoproline linked to L-Pro linked to Pro-Lys or Pro-Arg, or D-Pro linked to L-β-homoproline-Lys or L-β-homoproline-Arg; L-Asp or D-Asp are optionally linked at the n-terminus to RB, wherein RB is -(PEG 11)-GYIPEAPRDGQAYVRKDGEWVLLSTFL, or -(PEG 11)-(Gly-Pro-HydroxyPro)₁₀, (S)-2-amino-4-phenylbutanoic acid is linked to RC, wherein RC is D-Pro linked to

ProLys or ProArg, or D-Pro linked to L-β-homoproline-Lys or L-β-homoproline-L-Arg;
A10 is L-Cys, L-Ser, L-Ala, D-Cys, D-Ser, or D-Ala;
the carboxy-terminal amino acid is in amide or carboxy- form;
at least one sulfhydryl amino acid is present as one of the amino acids in the sequence; and
A1, A2, A9, A10, or a combination thereof are optionally absent.

A mini-hepcidin of formula A1-A2-A3-A4-A5-A6-A7-A8-A9-A10 or A10-A9-A8-A7-A6-A5-A4-A3-A2-A1 may be a cyclic peptide or a linear peptide.

For example, A1 may be L-Asp; A2, may be L-Th; A3 may be L-His; A4 may be L-Phe; A5 may be L-Pro; A6 may be L-Ile; A7 may be L-Cys, D-Cys, S-t-butylthio-L-cysteine, L-homocysteine, L-penicillamine, or D-penicillamine; A8 may be L-Ile; A9 may be L-Phe; A10 may be absent; and the C-terminus may be amidated. Alternatively, A3 may be L-His; A4 may be L-Phe; A5 may be L-Pro; A6 may be L-Ile; A7 may be L-Cys, D-Cys, S-t-butylthio-L-cysteine, L-homocysteine, L-penicillamine, or D-penicillamine; A8 may be L-Ile; A1, A2, A9, and A10 may be absent, and the C-terminus may be amidated. Alternatively, A3 may be L-His; A4 may be L-Phe; A5 may be L-Pro; A6 may be L-Ile; A7 may be L-Cys, D-Cys, S-t-butylthio-L-cysteine, L-homocysteine, L-penicillamine, or D-penicillamine; A1, A2, A8, A9, and A10 may be absent; and the C-terminus may be amidated.

A mini-hepcidin may comprise the amino acid sequence HFPICI (SEQ ID NO:11), HFPICIF (SEQ ID NO:12), DTHFPICIDTHFPICIF (SEQ ID NO:13), DTHFPIAIFC (SEQ ID NO:14), DTHAPICIF (SEQ ID NO:15), DTHFPICIF (SEQ ID NO:16), or CDTHFPICIF (SEQ ID NO:17). The mini-hepcidin may comprise the sequence set forth in SEQ ID NO:15, for example, wherein the cysteine forms a disulfide bond with S-tertbutyl.

A mini-hepcidin may comprise the amino acid sequence D-T-H-F-P-I-(L-homocysteine)-I-F; D-T-H-F-P-I-(L-penicillamine)-I-F; D-T-H-F-P-I-(D-penicillamine)-I-F; D-(L-tert-leucine)-H-(L-phenylglycine)-(octahydroindole-2-carboxylic acid)-(L-α-cyclohexylglycine)-C-(L-α-cyclohexylglycine)-F; or D-(L-tert-leucine)-H-P-(octahydroindole-2-carboxylic acid)-(L-α-cyclohexylglycine)-C-(L-α-cyclohexylglycine)-F.

A mini-hepcidin may comprise the amino acid sequence FICIPFHTD (SEQ ID NO:18), FICIPFH (SEQ ID NO:19), R2-FICIPFHTD (SEQ ID NO:20), R3-FICIPFHTD (SEQ ID NO:21), FICIPFHTD-R6 (SEQ ID NO:22), R4-FICIPFHTD (SEQ ID NO:23), or R5-FICIPFHTD (SEQ ID NO:24), wherein each amino acid is a D amino acid; R1 is —CONH₂—CH₂—CH₂—S; R2 is chenodeoxycholate-(PEG 11)-; R3 is ursodeoxycholate-(PEG11)-; R4 is palmitoyl-(PEG11)-; R5 is 2(palmitoyl)-diaminopropionic acid-(PEG 11)-; and R6 is (PEG 11)-GYIPEAPRDGQAYVRKDGEWVLLSTFL, wherein each amino acid of R6 is an L amino acid.

A mini-hepcidin may comprise the amino acid sequence D-T-H-((S)-2-amino-4-phenylbutanoic acid)-P-I-C-I-F; D-T-H-(3,3-diphenyl-L-alanine)-P-I-C-I-F; D-T-H-(L-biphenylalanine)-P-I-C-I-F; D-T-H-((1-naphthyl)-L-alanine)-P-I-C-I-F; D-T-H-((S)-3-amino-4,4-diphenylbutanoic acid)-P-I-C-I-F; D-T-H-F-P-I-C-I-((S)-2-amino-4-phenylbutanoic acid); D-T-H-F-P-I-C-I-(3,3-diphenyl-L-alanine); D-T-H-F-P-I-C-I-(L-biphenylalanine); D-T-H-F-P-I-C-I-((1-naphthyl)-L-alanine); D-T-H-F-P-I-C-I-((S)-3-amino-4,4-diphenylbutanoic acid); D-T-H-(3,3-diphenyl-L-alanine)-P-I-C-I-(3,3-diphenyl-L-alanine); D-(3,3-diphenyl-L-alanine)-P-I-C-I-F; D-(3,3-diphenyl-L-alanine)-P-I-C-I-(3,3-diphenyl-L-alanine); D-T-H-(3,3-diphenyl-L-alanine)-P-R-C-R-(3,3-diphenyl-L-alanine); D-T-H-(3,3-diphenyl-L-alanine)-(octahydroindole-2-carboxylic acid)-I-C-I-F; D-T-H-(3,3-diphenyl-L-alanine)-(octahydroindole-2-carboxylic acid)-I-C-I-(3,3-diphenyl-L-alanine); or D-T-H-(3,3-diphenyl-L-alanine)-P-C-C-C-(3,3-diphenyl-L-alanine).

A mini-hepcidin may comprise the amino acid sequence D-T-H-F-P-I-C-I-F-R8; D-T-H-F-P-I-C-I-F-R9; D-T-H-F-P-I-C-I-F-R10; D-T-H-F-P-I-C-I-F-R11; D-T-H-F-P-I-C-I-F-R12; D-T-H-F-P-I-C-I-F-R13; D-T-H-F-P-I-C-I-((S)-2-amino-4-phenylbutanoic acid)-R8; D-T-H-F-P-I-C-I-((S)-2-amino-4-phenylbutanoic acid)-R9; D-T-H-F-P-I-C-I-((S)-2-amino-4-phenylbutanoic acid)-R12; or D-T-H-F-P-I-C-I-((S)-2-amino-4-phenylbutanoic acid)-R13, wherein R8 is D-Pro-L-Pro-L-Lys; R9 is D-Pro-L-Pro-L-Arg; R10 is (L-β-homoproline)-L-Pro-L-Lys; R11 is (L-β-homoproline)-L-Pro-L-Arg; R12 is D-Pro-(L-β-homoproline)-L-Lys; and R13 is D-Pro-(L-β-homoproline)-L-Arg.

A mini-hepcidin may comprise the amino acid sequence D-T-H-(3,3-diphenyl-L-alanine)-P-(D)R-C-(D)R-(3,3-diphenyl-L-alanine).

A mini-hepcidin may comprise the amino acid sequence C-(isonipecotic acid)-(3,3-diphenyl-D-alanine)-(4-(aminomethyl)cyclohexane carboxylic acid)-R-(4-(aminomethyl)cyclohexane carboxylic acid)-(isonipecotic acid)-(3,3-diphenyl-L-alanine)-cysteamide. A mini-hepcidin may comprise the amino acid sequence C-P-(3,3-diphenyl-D-alanine)-(4-(aminomethyl)cyclohexane carboxylic acid)-R-(4-(aminomethyl)cyclohexane carboxylic acid)-(isonipecotic acid)-(3,3-diphenyl-L-alanine)-cysteamide. A mini-hepcidin may comprise the amino acid sequence C-(D)P-(3,3-diphenyl-D-alanine)-(4-(aminomethyl)cyclohexane carboxylic acid)-R-(4-(aminomethyl)cyclohexane carboxylic acid)-(isonipecotic acid)-(3,3-diphenyl-L-alanine)-cysteamide. A mini-hepcidin may comprise the amino acid sequence C-G-(3,3-diphenyl-D-alanine)-(4-(aminomethyl)cyclohexane carboxylic acid)-R-(4-(aminomethyl)cyclohexane carboxylic acid)-(isonipecotic acid)-(3,3-diphenyl-L-alanine)-cysteamide.

A mini-hepcidin may comprise the amino acid sequence (2,2′-azanediyldiacetic acid)-Thr-His-(3,3-diphenyl-L-alanine)-(L-β-homoproline)-Arg-Cys-Arg-((S)-2-amino-4-phenylbutanoic acid)-(aminohexanoic acid)-(2,2′-azanediyldiacetic acid having a palmitylamine amide on the side chain), which is described in U.S. Pat. No. 9,328,140 (e.g., SEQ ID NO:94 of the '140 patent; hereby incorporated by reference).

In some embodiments, a mini-hepcidin has about 10% to 1000% of the activity of a 25 amino acid long peptide comprising the amino acid sequence set forth in SEQ ID NO:1. For example, a mini-hepcidin may have about 50% to about 200% of the activity of a 25 amino acid long peptide comprising the amino acid sequence set forth in SEQ ID NO:1, such as about 75% to about 150% of the activity, about 80% to about 120% of the activity, about 90% to about 110% of the activity, or about 95% to about 105% of the activity. The term “activity” may refer to the ability of a mini-hepcidin to specifically bind to ferroportin, e.g., thereby inhibiting the transport of intracellular iron into the extracellular space, inhibiting the absorption of dietary iron, and/or reducing serum iron concentration. Activity may refer to the ability of a mini-hepcidin to inhibit the transport of intracellular iron into the extracellular space. Activity may refer to the ability of a mini-hepcidin to inhibit the absorption of dietary iron. Activity may refer to the ability of a mini-hepcidin to reduce serum iron concentration in vivo.

### V. Routes of Administration

The compositions of the invention can be administered in a variety of conventional ways. In some aspects, the compositions of the invention are suitable for parenteral administration. These compositions may be administered, for example, intraperitoneally, intravenously, intrarenally, or intrathecally. In some aspects, the compositions of the invention are injected intravenously.

The composition may be administered topically, enterally, or parenterally. The composition may be administered subcutaneously, intravenously, intramuscularly, intranasally, by inhalation, orally, sublingually, by buccal administration, topically, transdermally, or transmucosally. The composition may be administered by injection. In preferred embodiments, the composition is administered by subcutaneous injection, orally, intranasally, by inhalation, or intravenously. In certain preferred embodiments, the composition is administered by subcutaneous injection.

Throughout this specification, the word “comprise” or variations such as “comprises” or “comprising” will be understood to imply the inclusion of a stated integer (or components) or group of integers (or components), but not the exclusion of any other integer (or components) or group of integers (or components). The singular forms “a,” “an,” and “the” include the plurals unless the context clearly dictates otherwise. The term “including” is used to mean “including but not limited to.” “Including” and “including but not limited to” are used interchangeably. The terms “patient” and “individual” are used interchangeably and refer to either a human or a non-human animal. These terms include mammals such as humans, primates, livestock animals (e.g., bovines, porcines), companion animals (e.g., canines, felines) and rodents (e.g., mice, rabbits and rats).

“About” and “approximately” shall generally mean an acceptable degree of error for the quantity measured given the nature or precision of the measurements. Typically, exemplary degrees of error are within 20%, preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

As used herein, the term “administering” means providing a pharmaceutical agent or composition to a subject, and includes, but is not limited to, administering by a medical professional and self-administering. Such an agent, for example, may be hepcidin, a mini-hepcidin, or a hepcidin analogue.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As used herein, a therapeutic that “prevents” a condition (e.g., iron overload) refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

In certain embodiments, agents of the invention may be used alone or conjointly administered with another type of therapeutic agent. As used herein, the phrase “conjoint administration” refers to any form of administration of two or more different therapeutic agents such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, or about a week of one another. Thus, a subject who receives such treatment can benefit from a combined effect of different therapeutic agents.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

The invention now being generally described, it will be more readily understood by reference to the following examples which are included merely for purposes of illustration of certain aspects and embodiments of the present invention, and are not intended to limit the invention.

### EXEMPLIFICATION

### Example 1

A study was designed to evaluate the effect of subcutaneous doses of hepcidin on serum iron levels in mice (n=6-7/group). When injected subcutaneously, a 50 μg dose of hepcidin showed a significant decrease in serum iron levels at 4 hours post dose (average of 40% decrease compared to vehicle, p<0.05), and 24 hours post dose (average of 15% decrease compared to vehicle, p<0.05).

### Example 2

A study was designed to evaluate doses of 50, 100, and 200 μg of hepcidin delivered subcutaneously and their effect on serum iron levels in mice (n=7/group). All three doses showed a significant decrease in serum iron levels at 4 hours post dose compared to vehicle (p<0.01). Conversely, 50 μg and 100 μg doses were elevated (p<0.01) compared to the vehicle at 24 hours post dose. The elevated levels of serum iron could be due to the system's reaction to the clearance of hepcidin. One mouse died following the 4-hour blood collection. Mortality was likely related to the stress of the blood collection. Serum iron levels normalized 72 hours post dose.

### Example 3

A study was designed to evaluate doses of 1, 5, 10, and 50 mg of hepcidin delivered subcutaneously and their effect on serum iron levels in normal rats (n=7/group). A significant decrease in serum iron levels was observed at all dose levels, and animals dosed at 50 mg still demonstrated an effect at 72 hours. Tₘₐₓ and Cₘₐₓ were reached between 1 and 2 hours post dose for all dose groups, but the uptake between the high and mid dose were very similar at these time points. No lethargy was observed in this study at any dose level. The lowest serum iron concentrations were observed at 4 hours post dose for all three doses. In the 5 mg dose, serum iron levels returned to pre-dose levels at 48 hours post dose. In the 10 mg and 50 mg dose groups, serum iron levels continued to increase, but did not return to pre-dose levels 72 hours post dose.

### Example 4

Hepcidin was evaluated in two expanded, acute studies in rats and dogs. These studies were conducted to determine the no-observed adverse effect level (NOAEL). The NOAEL was determined to be 5 mg/kg/day in dogs due to various clinical and histopathological observations.

A study was designed to evaluate doses of 5, 25, and 50 mg/kg of hepcidin (human equivalent dose of 0.8, 4, 8 mg/kg, respectively), delivered SC to Sprague Dawley rats (n=9/sex/group). All doses showed significantly decreased average serum iron levels when compared to vehicle and their pre-dose levels. The lowest serum iron level was observed at 4 hours post dose for all three doses. No unexpected adverse effects were observed in this study. Hepcidin-related changes were limited to non-adverse, dose-independent, reductions in food consumption and body weight gain, and induration at the injection site. As would be anticipated with the administration of hepcidin, biological effects observed included dose-dependent reversible decreases in reticulocytes and iron concentration, and increased unsaturated iron binding capacity. On average, the female rat serum iron levels were observed to be higher, but the toxicokinetic (TK) effect of hepcidin was comparable for both sexes. The results demonstrate that hepcidin is able to decrease serum iron levels significantly in Sprague Dawley rats without unexpected physiological changes to any major organs. The clinical pathology and iron-related changes were consistent with the expected pharmacology of hepcidin. Based on these results, the NOAEL was determined to be 50 mg/kg/day.

A study was designed to evaluate doses of 5, 25, and 50 mg/kg (human equivalent dose of 0.8, 4, and 8 mg/kg, respectively), of hepcidin delivered in a single subcutaneous dose to dogs (n=6/sex/group). Increased thickness in the administration site was observed on Day 4 at 50 mg/kg and on Day 15 at ≥25 mg/kg. Microscopic findings on Day 4 consisted of mixed cell infiltration in the administration site in males and females at ≥25 mg/kg, while on Day 15, microscopic findings at the administration site included mixed cell infiltration in males and females at ≥5 mg/kg, fibrosis in males at ≥25 mg/kg and in females at ≥5 mg/kg, and cystic space in males at 50 mg/kg and in females at ≥25 mg/kg. Based on these results, the NOAEL was considered to be 5 mg/kg/day. The testing showed temporary increases in neutrophils and fibrinogen levels up to Day 4 in ≥25 mg/kg/day dose groups. Although these blood chemistry analytes were temporarily increased, they were not considered serious, and the NOAEL dose was determined to be 5 mg/kg/day at the conclusion of this study. Other adverse reactions were as follows: hunched posture, soft feces, gross pathology finding of “thick,” and subcutaneous fibrosis, mixed cell infiltration, and cysts present at recovery period.

### INCORPORATION BY REFERENCE

All publications and patents mentioned herein are hereby incorporated by reference in their entirety as if each individual publication or patent was specifically and individually indicated to be incorporated by reference. In case of conflict, the present specification, including its specific definitions, will control. While specific aspects of the patient matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

What is claimed is:

1. A method of treating and/or preventing acute kidney injury in a subject by administering a composition comprising hepcidin or mini-hepcidin to the subject.

2. A method of treating and/or preventing insulin resistance in a subject by administering a composition comprising hepcidin or mini-hepcidin to the subject.

4. A method of treating and/or preventing a condition associated with reduced iron absorption by bone marrow in a subject by administering a composition comprising hepcidin or mini-hepcidin to the subject.

3. A method of treating and/or preventing insulin insufficiency in a subject by administering a composition comprising hepcidin or mini-hepcidin to the subject.

5. A method for treating acquired iron overload in a subject, comprising administering a composition comprising hepcidin or mini-hepcidin to the subject.

6. The method of claim 5, wherein the subject has received a blood transfusion within the past week.

7. The method of claim 5 or claim 6, wherein the subject has received at least five blood transfusions within the past year.

8. The method of any one of claims 5 to 7, wherein the subject has received at least ten blood transfusions within the past year.

9. A method for preventing iron overload in a subject undergoing a blood transfusion comprising administering a composition comprising hepcidin or mini-hepcidin to the subject.

10. The method of claim 9, wherein the composition is administered to the subject before the subject undergoes the blood transfusion.

11. The method of claim 10, wherein the composition is administered to the subject no more than 1 day before the subject undergoes the blood transfusion.

12. The method of claim 11, wherein the composition is administered to the subject no more than 6 hours before the subject undergoes the blood transfusion.

13. The method of claim 11, wherein the composition is administered to the subject no more than 1 hour before the subject undergoes the blood transfusion.

14. The method of claim 9, wherein the composition is administered to the subject while the subject is undergoing the blood transfusion.

15. The method of claim 9, wherein the composition is administered to the subject after the subject has undergone the blood transfusion, e.g., within about an hour, within about two hours, within about six hours, within about twelve hours, or within about one day after the transfusion.

16. The method of claim 15, wherein the composition is administered to the subject no more than 1 week after the subject has undergone the blood transfusion.

17. The method of claim 15, wherein the composition is administered to the subject no more than 3 days after the subject has undergone the blood transfusion.

18. The method of claim 15, wherein the composition is administered to the subject no more than 1 day after the subject has undergone the blood transfusion.

19. The method of any one of claims 5 to 18, wherein the subject has anemia.

20. The method of claim 19, wherein the anemia is aplastic anemia, hemolytic anemia, or sideroblastic anemia.

21. The method of any one of claims 5 to 20, wherein the subject has thalassemia, sickle cell disease, or myelodysplastic syndrome.

22. The method of any one of claims 6 to 18, wherein the subject was administered a blood transfusion after suffering a physical trauma.

23. The method of any one of claims 1 to 22, wherein administering a composition to the subject comprises administering about 10 μg to about 1 gram of hepcidin or mini-hepcidin.

24. The method of claim 23, wherein administering a composition to the subject comprises administering about 100 μg to about 100 mg of hepcidin or mini-hepcidin.

25. The method of claim 24, wherein administering a composition to the subject comprises administering about 200 μg to about 50 mg of hepcidin or mini-hepcidin.

26. The method of claim 25, wherein administering a composition to the subject comprises administering about 500 μg to about 10 mg of hepcidin or mini-hepcidin.

27. The method of claim 26, wherein administering a composition comprising hepcidin or mini-hepcidin to the subject comprises administering about 500 μg, about 600 μg, about 667 μg, about 700 μg, about 750 μg, about 800 μg, about 850 μg, about 900 μg, about 950 μg, about 1000 μg, about 1200 μg, about 1250 μg, about 1300 μg, about 1333 μg, about 1350 μg, about 1400 μg, about 1500 μg, about 1667 μg, about 1750 μg, about 1800 μg, about 2000 μg, about 2200 μg, about 2250 μg, about 2300 μg, about 2333 μg, about 2350 μg, about 2400 μg, about 2500 μg, about 2667 μg, about 2750 μg, about 2800 μg, about 3 mg, about 3.3 mg, about 3.5 mg, about 3.7 mg, about 4 mg, about 4.5 mg, about 5 mg, about 6 mg, about 7 mg, about 8 mg, about 9 mg, or about 10 mg of hepcidin or mini-hepcidin.

28. The method of any one of claims 1 to 27, wherein the composition is administered subcutaneously, intravenously, intramuscularly, intranasally, by inhalation, orally, sublingually, by buccal administration, topically, transdermally, or transmucosally.

29. The method of any one of claims 1 to 28, wherein the composition is administered by injection.

30. The method of claim 28, wherein the composition is administered intravenously.

31. The method of any one of the preceding claims, wherein the subject is a mammal.

32. The method of claim 31, wherein the subject is a rodent, lagomorph, feline, canine, porcine, ovine, bovine, equine, or primate.

33. The method of claim 32, wherein the subject is a human.

34. The method of any one of claims 1 to 33, wherein the subject has a total body iron content of about 40 to about 50 mg/kg prior to administering the composition.

35. The method of any one of claims 1 to 33, wherein the subject has a total body iron content greater than 50 mg/kg prior to administering the composition.

36. The method of claim 35, wherein the subject has a total body iron content greater than 60 mg/kg prior to administering the composition.

37. The method of any one of claims 1 to 36, wherein the serum iron concentration of the subject is at least about 100 μg/dL prior to administering the composition.

38. The method of claim 37, wherein the serum iron concentration of the subject is at least about 200 μg/dL prior to administering the composition.

39. The method of any one of claims 1 to 38, wherein the transferrin saturation of the subject is greater than about 20% prior to administering the composition to the subject.

40. The method of claim 39, wherein the transferrin saturation of the subject is greater than about 50% prior to administering the composition to the subject.

41. The method of any one of claims 1 to 40, wherein the composition comprises hepcidin and the hepcidin comprises the amino acid sequence set forth in SEQ ID NO:1, SEQ ID NO:2, SEQ ID NO:3, SEQ ID NO:4, or SEQ ID NO:5.

42. The method of any one of claims 1 to 40, wherein the composition comprises hepcidin and the hepcidin comprises an amino acid sequence having at least 90% sequence homology with the amino acid sequence set forth in SEQ ID NO:1, SEQ ID NO:2, SEQ ID NO:3, SEQ ID NO:4, or SEQ ID NO:5.

43. The method of claim 42, wherein the hepcidin comprises each of the 8 cysteines in SEQ ID NO:1, SEQ ID NO:2, SEQ ID NO:3, SEQ ID NO:4, or SEQ ID NO:5.

44. The method of claim 43, wherein the 8 cysteines in SEQ ID NO:1, SEQ ID NO:2, SEQ ID NO:3, SEQ ID NO:4, or SEQ ID NO:5 form 4 disulfide bonds in the hepcidin.

45. The method of any one of claims 1 to 40, wherein the hepcidin comprises the amino acid sequence set forth in SEQ ID NO:1.

46. The method of any one of claims 1 to 40, wherein the composition comprises hepcidin and the hepcidin comprises the sequence set forth in SEQ ID NO:6, SEQ ID NO:7, SEQ ID NO:8, SEQ ID NO:9, or SEQ ID NO:10.

47. The method of claim 46, wherein the 8 cysteines of SEQ ID NO:6, SEQ ID NO:7, SEQ ID NO:8, SEQ ID NO:9, or SEQ ID NO:10 form 4 disulfide bonds in the hepcidin.

48. The method of any one of claims 1 to 36, wherein the composition comprises a mini-hepcidin.

49. The method of any one of claims 1 to 44, comprising administering hepcidin or mini-hepcidin conjointly with an iron chelation therapy and/or an iron-deficient diet.

50. A method for treating and/or preventing a condition in a subject, comprising administering a composition comprising hepcidin or mini-hepcidin to the subject.

51. The method of claim 50, wherein the condition is acute coronary syndrome or sepsis.

52. The method of claim 50, wherein the condition is iron overload, and the subject is undergoing cardiovascular surgery such as a cardiopulmonary bypass.

53. The method of claim 50, wherein the condition is iron overload, and the subject has undergone cardiovascular surgery such as a cardiopulmonary bypass, e.g., within about an hour, within about two hours, within about six hours, within about twelve hours, or within about one day after the transfusion.

54. The method of any one of claims 50 to 53, wherein administering a composition to the subject comprises administering about 10 μg to about 1 gram of hepcidin or mini-hepcidin.

55. The method of claim 54, wherein administering a composition to the subject comprises administering about 100 μg to about 100 mg of hepcidin or mini-hepcidin.

56. The method of claim 55, wherein administering a composition to the subject comprises administering about 200 μg to about 50 mg of hepcidin or mini-hepcidin.

57. The method of claim 56, wherein administering a composition to the subject comprises administering about 500 μg to about 10 mg of hepcidin or mini-hepcidin.

58. The method of claim 57, wherein administering a composition comprising hepcidin or mini-hepcidin to the subject comprises administering about 500 μg, about 600 μg, about 667 μg, about 700 μg, about 750 μg, about 800 μg, about 850 μg, about 900 μg, about 950 μg, about 1000 μg, about 1200 μg, about 1250 μg, about 1300 μg, about 1333 μg, about 1350 μg, about 1400 μg, about 1500 μg, about 1667 μg, about 1750 μg, about 1800 μg, about 2000 μg, about 2200 μg, about 2250 μg, about 2300 μg, about 2333 μg, about 2350 μg, about 2400 μg, about 2500 μg, about 2667 μg, about 2750 μg, about 2800 μg, about 3 mg, about 3.3 mg, about 3.5 mg, about 3.7 mg, about 4 mg, about 4.5 mg, about 5 mg, about 6 mg, about 7 mg, about 8 mg, about 9 mg, or about 10 mg of hepcidin or mini-hepcidin.

59. The method of any one of claims 50 to 58, wherein the composition is administered subcutaneously, intravenously, intramuscularly, intranasally, by inhalation, orally, sublingually, by buccal administration, topically, transdermally, or transmucosally.

60. The method of any one of claims 46 to 55, wherein the composition is administered by injection.

61. The method of claim 59, wherein the composition is administered intravenously.

62. The method of any one of claims 50 to 61, wherein the composition comprises hepcidin and the hepcidin comprises the amino acid sequence set forth in SEQ ID NO:1, SEQ ID NO:2, SEQ ID NO:3, SEQ ID NO:4, or SEQ ID NO:5.

63. The method of any one of claims 50 to 61, wherein the composition comprises hepcidin and the hepcidin comprises an amino acid sequence having at least 90% sequence homology with the amino acid sequence set forth in SEQ ID NO:1, SEQ ID NO:2, SEQ ID NO:3, SEQ ID NO:4, or SEQ ID NO:5.

64. The method of claim 63, wherein the hepcidin comprises each of the 8 cysteines in SEQ ID NO:1, SEQ ID NO:2, SEQ ID NO:3, SEQ ID NO:4, or SEQ ID NO:5.

65. The method of claim 64, wherein the 8 cysteines in SEQ ID NO:1, SEQ ID NO:2, SEQ ID NO:3, SEQ ID NO:4, or SEQ ID NO:5 form 4 disulfide bonds in the hepcidin.

66. The method of any one of claims 50 to 65, wherein the hepcidin comprises the amino acid sequence set forth in SEQ ID NO:1.

67. The method of any one of claims 50 to 61, wherein the composition comprises hepcidin and the hepcidin comprises the sequence set forth in SEQ ID NO:6, SEQ ID NO:7, SEQ ID NO:8, SEQ ID NO:9, or SEQ ID NO:10.

68. The method of claim 67, wherein the 8 cysteines of SEQ ID NO:6, SEQ ID NO:7, SEQ ID NO:8, SEQ ID NO:9, or SEQ ID NO:10 form 4 disulfide bonds in the hepcidin.

69. The method of any one of claims 50 to 68, wherein the composition comprises a mini-hepcidin.

70. The method of any one of claims 50 to 69, comprising administering hepcidin or mini-hepcidin conjointly with an iron chelation therapy and/or an iron-deficient diet.

71. A method of reducing, preventing, or reversing organ damage or enhancing organ preservation comprising administering a composition comprising hepcidin or mini-hepcidin to an organ donor prior to removal of the organ.

72. The method of claim 71, wherein the composition comprising hepcidin or mini-hepcidin is administered to the organ donor less than 24 hours prior to removal of the organ.

73. The method of claim 71, wherein the composition comprising hepcidin or mini-hepcidin is administered to the organ donor less than 1 hour prior to removal of the organ.

74. A method of reducing, preventing, or reversing organ damage or enhancing organ preservation comprising contacting the organ with a preservation solution wherein the preservation solution comprises a composition comprising hepcidin or mini-hepcidin.

75. A method of facilitating an organ transplant procedure or enhancing the success of an organ transplant procedure, comprising contacting the organ ex vivo with a preservation solution wherein the preservation solution comprises a composition comprising hepcidin or mini-hepcidin.

76. A method of prolonging organ viability ex vivo, comprising contacting the organ ex vivo with a preservation solution wherein the preservation solution comprises a composition comprising hepcidin or mini-hepcidin.

77. The method of any one of claims 71 to 76, wherein the composition comprises hepcidin and the hepcidin comprises the amino acid sequence set forth in SEQ ID NO:1, SEQ ID NO:2, SEQ ID NO:3, SEQ ID NO:4, or SEQ ID NO:5.

78. The method of any one of claims 71 to 76, wherein the composition comprises hepcidin and the hepcidin comprises an amino acid sequence having at least 90% sequence homology with the amino acid sequence set forth in SEQ ID NO:1, SEQ ID NO:2, SEQ ID NO:3, SEQ ID NO:4, or SEQ ID NO:5.

79. The method of claim 78, wherein the hepcidin comprises each of the 8 cysteines in SEQ ID NO:1, SEQ ID NO:2, SEQ ID NO:3, SEQ ID NO:4, or SEQ ID NO:5.

80. The method of claim 79, wherein the 8 cysteines in SEQ ID NO:1, SEQ ID NO:2, SEQ ID NO:3, SEQ ID NO:4, or SEQ ID NO:5 form 4 disulfide bonds in the hepcidin.

81. The method of any one of claims 71 to 76, wherein the hepcidin comprises the amino acid sequence set forth in SEQ ID NO:1.

82. The method of any one of claims 71 to 76, wherein the composition comprises hepcidin and the hepcidin comprises the sequence set forth in SEQ ID NO:6, SEQ ID NO:7, SEQ ID NO:8, SEQ ID NO:9, or SEQ ID NO:10.

83. The method of claim 82, wherein the 8 cysteines of SEQ ID NO:6, SEQ ID NO:7, SEQ ID NO:8, SEQ ID NO:9, or SEQ ID NO:10 form 4 disulfide bonds in the hepcidin.

84. The method of any one of claims 71 to 76, wherein the composition comprises a mini-hepcidin.

85. A method of treating and/or preventing a condition in a subject by administering a composition comprising hepcidin or mini-hepcidin to the subject, wherein the condition is insulin resistance, insulin insufficiency, carotid artery lesion, chronic kidney disease, acute kidney injury, proteinuria, anti-glomerular basement membrane (anti-GMB) glomerulonephritis, minimal change disease, membrane nephropathy, autoimmune glomerulonephritis, or a condition associated with reduced iron absorption by bone marrow.

86. The method of claim 85, wherein the condition is insulin resistance.

87. The method of claim 85, wherein the condition is insulin insufficiency.

88. The method of claim 85, wherein the condition is carotid artery lesion.

89. The method of claim 85, wherein the condition is chronic kidney disease.

90. The method of claim 85, wherein the condition is acute kidney disease.

91. The method of claim 85, wherein the condition is proteinuria.

92. The method of claim 85, wherein the condition is anti-glomerular basement membrane (anti-GMB) glomerulonephritis.

93. The method of claim 85, wherein the condition is minimal change disease.

94. The method of claim 85, wherein the condition is membrane nephropathy.

95. The method of claim 85, wherein the condition is autoimmune glomerulonephritis.

96. The method of claim 85, wherein the condition is a condition associated with reduced iron absorption by bone marrow.

97. The method of any one of claims 85 to 96, wherein the subject has a total body iron content of about 40 to about 50 mg/kg prior to administering the composition.

98. The method of any one of claims 85 to 96, wherein the subject has a total body iron content greater than 50 mg/kg prior to administering the composition.

99. The method of claim 98, wherein the subject has a total body iron content greater than 60 mg/kg prior to administering the composition.

100. The method of claim 85, wherein the condition is caused by iron overload, such as an acquired iron overload, in the subject.

101. The method of claim 100, wherein the iron overload in the subject results from a blood transfusion, a cardiovascular surgery, cardiopulmonary bypass, an acute coronary syndrome, or sepsis.

102. The method of any one of claims 85 to 101, wherein administering a composition to the subject comprises administering about 10 μg to about 1 gram of hepcidin or mini-hepcidin.

103. The method of claim 102, wherein administering a composition to the subject comprises administering about 100 μg to about 100 mg of hepcidin or mini-hepcidin.

104. The method of claim 103, wherein administering a composition to the subject comprises administering about 200 μg to about 50 mg of hepcidin or mini-hepcidin.

105. The method of claim 104, wherein administering a composition to the subject comprises administering about 500 μg to about 10 mg of hepcidin or mini-hepcidin.

106. The method of claim 105, wherein administering a composition comprising hepcidin or mini-hepcidin to the subject comprises administering about 500 μg, about 600 μg, about 667 μg, about 700 μg, about 750 μg, about 800 μg, about 850 μg, about 900 μg, about 950 μg, about 1000 μg, about 1200 μg, about 1250 μg, about 1300 μg, about 1333 μg, about 1350 μg, about 1400 μg, about 1500 μg, about 1667 μg, about 1750 μg, about 1800 μg, about 2000 μg, about 2200 μg, about 2250 μg, about 2300 μg, about 2333 μg, about 2350 μg, about 2400 μg, about 2500 μg, about 2667 μg, about 2750 μg, about 2800 μg, about 3 mg, about 3.3 mg, about 3.5 mg, about 3.7 mg, about 4 mg, about 4.5 mg, about 5 mg, about 6 mg, about 7 mg, about 8 mg, about 9 mg, or about 10 mg of hepcidin or mini-hepcidin.

107. The method of any one of claims 85 to 106, wherein the composition is administered subcutaneously, intravenously, intramuscularly, intranasally, by inhalation, orally, sublingually, by buccal administration, topically, transdermally, or transmucosally.

108. The method of any one of claims 85 to 106, wherein the composition is administered by injection.

109. The method of claim 108, wherein the composition is administered intravenously.

110. The method of any one of claims 85 to 109, wherein the composition comprises hepcidin and the hepcidin comprises the amino acid sequence set forth in SEQ ID NO:1, SEQ ID NO:2, SEQ ID NO:3, SEQ ID NO:4, or SEQ ID NO:5.

111. The method of any one of claims 85 to 109, wherein the composition comprises hepcidin and the hepcidin comprises an amino acid sequence having at least 90% sequence homology with the amino acid sequence set forth in SEQ ID NO:1, SEQ ID NO:2, SEQ ID NO:3, SEQ ID NO:4, or SEQ ID NO:5.

112. The method of claim 111, wherein the hepcidin comprises each of the 8 cysteines in SEQ ID NO:1, SEQ ID NO:2, SEQ ID NO:3, SEQ ID NO:4, or SEQ ID NO:5.

113. The method of claim 111, wherein the 8 cysteines in SEQ ID NO:1, SEQ ID NO:2, SEQ ID NO:3, SEQ ID NO:4, or SEQ ID NO:5 form 4 disulfide bonds in the hepcidin.

114. The method of any one of claims 85 to 109, wherein the hepcidin comprises the amino acid sequence set forth in SEQ ID NO:1.

115. The method of any one of claims 85 to 109, wherein the composition comprises hepcidin and the hepcidin comprises the sequence set forth in SEQ ID NO:6, SEQ ID NO:7, SEQ ID NO:8, SEQ ID NO:9, or SEQ ID NO:10.

116. The method of claim 115, wherein the 8 cysteines of SEQ ID NO:6, SEQ ID NO:7, SEQ ID NO:8, SEQ ID NO:9, or SEQ ID NO:10 form 4 disulfide bonds in the hepcidin.

117. The method of any one of claims 85 to 109, wherein the composition comprises a mini-hepcidin.

118. The method of any one of claims 85 to 117, further comprising administering to the subject an iron chelation therapy and/or an iron-deficient diet.

119. A method of reducing total body iron in a subject by administering hepcidin or mini-hepcidin to the subject.

120. The method of claim 119, wherein the subject has acquired iron overload.

121. The method of claim 120, wherein the subject has acquired iron overload resulting from a blood transfusion.

122. The method of claim 120, wherein the subject has acquired iron overload resulting from a cardiovascular surgery, cardiopulmonary bypass, acute coronary syndrome, or sepsis.

123. The method of claim 119, wherein the subject has a condition, wherein the condition is insulin resistance, insufficiency (diabetes), carotid artery lesion, chronic kidney disease, acute kidney injury, proteinuria, anti-glomerular basement membrane (anti-GMB) glomerulonephritis, minimal change disease (nephrotic syndrome), membrane nephropathy, or autoimmune glomerulonephritis (e.g., immune complex induced glomerulonephritis).

124. The method of claim 123, wherein the condition is caused by acquired iron overload.

125. The method of any one of claims 119 to 124, comprising administering hepcidin or mini-hepcidin conjointly with an iron chelation therapy and/or an iron-deficient diet.

126. The method of any one of claims 119 to 124, comprising administering hepcidin or mini-hepcidin in the absence of an iron chelation therapy and/or an iron-deficient diet.

127. The method of any one of claims 119 to 124, consisting of administering hepcidin or mini-hepcidin to treat and/or prevent iron overload.

128. The method of any one of claims 119 to 124, comprising discontinuing an iron chelation therapy and/or an iron-deficient diet administered to the subject and commencing administering hepcidin or mini-hepcidin to the subject.

129. The method of claim 128, comprising discontinuing the iron chelation therapy and/or the iron-deficient diet administered to the subject and after one day, two days, three days, four days, five days, six days, seven days, eight days, nine days, ten days, eleven days, twelve days, thirteen days, or fourteen days, commencing administering hepcidin or mini-hepcidin to the subject.

130. The method of claim 124 or 125, comprising discontinuing the iron chelation therapy and/or the iron-deficient diet administered to the subject and after one day, two days, three days, four days, five days, six days, or seven days commencing administering hepcidin or mini-hepcidin to the subject.

131. The method of claim 128, comprising commencing administering hepcidin or mini-hepcidin to the subject who is receiving the iron chelation therapy and/or an iron-deficient diet and after one day, two days, three days, four days, five days, six days, seven days, eight days, nine days, ten days, eleven days, twelve days, thirteen days, or fourteen days, discontinuing the iron chelation therapy and/or the iron-deficient diet administered to the subject.

132. The method of claim 124 or 131, comprising commencing administering hepcidin or mini-hepcidin to the subject who is receiving the iron chelation therapy and/or an iron-deficient diet and after one day, two days, three days, four days, five days, six days, or seven days, discontinuing the iron chelation therapy and/or the iron-deficient diet administered to the subject.
